(19) United States
(12) Patent Application Publication
(10) Pub. No.: US 2009/0054428 A1
(43) Pub. Date: Feb. 26, 2009

(54) NOVEL PYRIMIDINE DERIVATIVES 965

(75) Inventors: Bernard Christophe Barlaam, Reims (FR); Richard Ducray, Reims (FR)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/175,010

(22) Filed: Jul. 17, 2008

(30) Foreign Application Priority Data
Jul. 19, 2007 (EP) 07301252.8

(51) Int. Cl. A61K 31/5377; C07D 413/14; A61P 35/00

(52) U.S. Cl. 514/232.2; 544/82

(57) ABSTRACT

The invention concerns compounds of Formula I, or a pharmaceutically acceptable salt thereof,

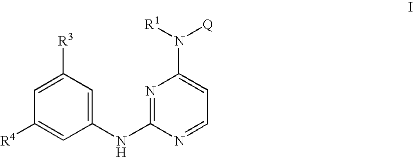

where R¹, Q, R³, and R⁴ are as defined in the description. The present invention also relates to processes for the preparation of such compounds, pharmaceutical compositions containing them and their use in the manufacture of a medicament for use as an antiproliferative agent in the prevention or treatment of tumours or other proliferative conditions which are sensitive to the inhibition of EphB4 kinases.

This application claims the benefit under 35 U.S.C. § 119(a)-(d) of Application No. 07301252.8 (EP) filed on 19 Jul. 2007.

The present invention relates to novel pyrimidine derivatives, to pharmaceutical compositions containing these derivatives and to their use in therapy, in particular in the prevention and treatment of cancer, in a warm blooded animal such as man.

Many of the current treatment regimes for cell proliferation diseases such as psoriasis and cancer utilise compounds which inhibit DNA synthesis. Such compounds are generally toxic to all cells, but their toxic effects on rapidly dividing cells, such as tumour cells, can be beneficial.

In recent years it has been discovered that a cell may become cancerous by virtue of the transformation of a portion of its DNA into an oncogene i.e. a gene which, on activation, leads to the formation of malignant tumour cells (Bradshaw, Mutagenesis, 1986, 1, 91). Several such oncogenes give rise to the production of peptides which are receptors for growth factors. Activation of the growth factor receptor results in an increase in cell proliferation. It is known, for example, that several oncogenes encode tyrosine kinase enzymes and that certain growth factor receptors are also tyrosine kinase enzymes (Yarden et al, Ann. Rev. Biochem., 1988, 57, 443; Larsen et al., Ann. Reports in Med. Chem. 1989, Chpt. 13).

Receptor tyrosine kinases play an important role in the transmission of biochemical signals, which initiate a variety of cell responses—including cell proliferation, survival and migration. They are large enzymes which span the cell membrane and possess an extracellular binding domain for growth factors, such as epidermal growth factor (EGF), and an intracellular portion which functions as a kinase to phosphorylate tyrosine amino acids in proteins and thereby influence cell proliferation. A large number of receptor tyrosine kinases are known (Wilks, Advances in Cancer Research, 1993, 60 43-73) and are classified on the basis of the family of growth factors that bind to the extracellular domain. This classification includes Class I receptor tyrosine kinases comprising the EGF family of receptor tyrosine kinases such as the EGF, TGFα, Neu and erbB receptors, Class II receptor tyrosine kinases comprising the insulin family of receptor tyrosine kinases such as the insulin and IGF1 receptors and insulin-related receptor (IRR), and Class III receptor tyrosine kinases comprising the platelet-derived growth factor (PDGF) family of receptor tyrosine kinases such as the PDGFα, PDGFβ and colony-stimulating factor 1 (CSF1) receptors.

The Eph family is the largest known family of receptor tyrosine kinases, with 14 receptors and 8 cognate ephrin ligands identified in mammals (reviewed in Kullander and Klein, Nature Reviews Molecular Cell Biology, 2002, 3, 475-486). The receptor family is further sub-divided into two sub-families defined largely by homology of extracellular domains and affinity towards a particular ligand type. In general, all Eph receptors contain an intracellular tyrosine kinase domain and an extracellular Ig-like domain with a cysteine-rich region with 19 conserved cysteines and two fibronectin type III domains. The A-class of Eph receptors consists of 8 receptors termed EphA1-8, which generally bind to their cognate ephrinA class of ligands termed ephrinA1-5. The B-class consists of 6 receptors termed EphB1-6, which bind to their cognate ephrinB ligands termed ephrinB1-3. Eph receptor ligands are unusual and differ to most other receptor tyrosine kinase ligands in that they are also tethered to cells, via a glycosylphosphatidylinositol linker in ephrinA ligands or an integral transmembrane region in ephrinB ligands. The binding of ephrin ligand to the Eph receptor induces a conformational change within the Eph intracellular domain that enables phosphorylation of tyrosine residues within an auto-inhibitory juxtamembrane region, which relieves this inhibition of catalytic site and enables additional phosphorylation to stabilise the active conformation and generate more docking sites for downstream signalling effectors.

Furthermore, evidence indicates that Eph/ephrin signalling can regulate other cell responses, such as proliferation and survival.

There is growing evidence that Eph receptor signalling may contribute to tumourigenesis in a wide variety of human cancers, either on tumour cells directly or indirectly via modulation of vascularisation. For instance, many Eph receptors are over-expressed in various tumour types (Reviewed in Surawska et al., Cytokine & Growth Factor Reviews, 2004, 15, 419-433, Nakamoto and Bergemann, Microscopy Res and Technique, 2002, 59, 58-67). The expression of EphB receptors, including EphB4, is up-regulated in tumours such as neuroblastomas, leukemias, breast, liver, lung and colon. Furthermore, various in vitro and in vivo studies particularly relating to EphB4 have indicated that over-expression of Eph receptors on cancer cells is able to confer tumourigenic phenotypes such as proliferation and invasion, consistent with the speculated role in oncogenesis.

For instance, inhibition of EphB4 expression using interfering-RNA or antisense oligodeoxynucleotides inhibited proliferation, survival and invasion of PC3 prostate cancer cells in vitro and in vivo xenograft model (Xia et al., Cancer Res., 2005, 65, 4623-4632.

In addition to compelling role of Eph receptors on tumour cells, there is good evidence that EphB4 may contribute to tumour vascularisation (Reviewed in Brantley-Sieders et al., Current Pharmaceutical Design, 2004, 10, 3431-3442, Cheng et al., Cytokine and Growth Factor Reviews, 2002, 13, 75-85). Members of Eph family including EphB4 are expressed on endothelial cells. Transgenic studies have shown that disruption of EphB4 (Gerety et al., Molecular Cell, 1999, 4, 403-414) or its ligand ephrinB2 (Wang et al., Cell, 1998, 93, 741-753) causes embryonic lethality associated with vascular modelling defects consistent with a critical role in vessel development. EphB4 activation stimulates endothelial cell proliferation and migration in vitro (Steinle et al., J. Biol. Chem., 2002, 277, 43830-43835).

Moreover, inhibition of EphB4 signalling using soluble extracellular-domains of EphB4 have been shown to inhibit tumour growth and angiogenesis in in vivo xenograft studies (Martiny-Baron et al., Neoplasia, 2004, 6, 248-257, Kertesz et al., Blood, 2005, Pre-published online).

Accordingly it has been recognised that an inhibitor of Eph receptors, particularly EphB4, should be of value as a selective inhibitor of the proliferation and survival of tumour cells by either targeting the tumour cells directly or via their effects on tumour vascularisation. Thus, such inhibitors should be valuable therapeutic agents for the containment and/or treatment of tumour disease.

The applicants have found that certain pyrimidine compounds are useful in the inhibition of EphB4 and therefore may be useful in therapy for the treatment of disease states in which increased EphB4 activity is implicated.

According to a first aspect of the invention, there is provided a compound of formula I

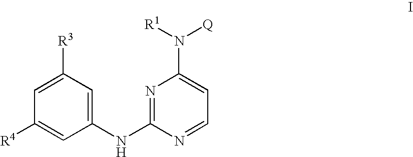

wherein:
R¹ is a (1-4C)alkyl group which is optionally substituted by one or more substituent groups selected from —OR⁵ (wherein R⁵ is selected from hydrogen or (1-2C)alkyl), cyano, halo, or —NR⁶R⁷ (where R⁶ and R⁷ are independently selected from hydrogen, (1-2C)alkyl or (1-2C)alkanoyl);
Q is selected from a group of formula:

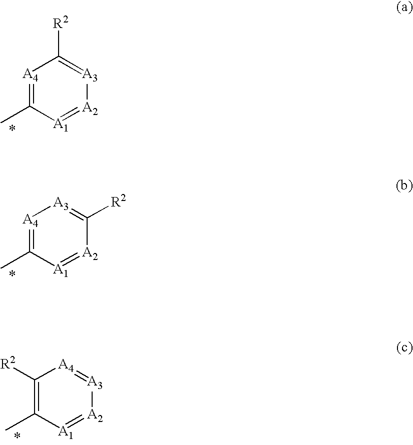

wherein * is the point of attachment to the compound of formula I above;
one of A₁, A₂, A₃, and A₄ is N and the others are —CR^{2a}—;
R² is independently selected from (1-2C)alkyl, (1-2C)alkoxy, fluoro, chloro, cyano, hydroxy(1-2C)alkyl, or a group of sub-formula:

—X¹—R^{y}

where X¹ is selected from —CO—, —NR^{a}—, —NR^{a}—CO—, —NR^{a}—COO—, NR^{a}CONR^{b}, —CONR^{a}—, —S(O)_{z}— (where z is 0, 1 or 2); —SO₂NR^{a}—, and —NR^{a}SO₂—, R^{a} and R^{b} are each independently selected from hydrogen or methyl, and R^{y} is hydrogen or (1-2C)alkyl;
each R^{2a} group present is independently selected from hydrogen, (1-2C)alkyl, (1-2C)alkoxy, fluoro, chloro, cyano, hydroxy(1-2C)alkyl, or a group of sub-formula:

—X²—R^{z}

where X² is selected from —CO—, —NR^{c}—, —NR^{c}—CO—, —NR^{c}—COO—, NR^{c}CONR^{C}, —CONR^{c}—, —S(O)_{z}— (where z is 0, 1 or 2); —SO₂NR^{c}—, and —NR^{c}SO₂—, R^{c} and R^{d} are each independently selected from hydrogen or methyl, and R^{z} is hydrogen or (1-2C)alkyl;
R³ is selected from:
(i) hydrogen, halo, nitro, cyano, or hydroxy;
(ii) an optionally substituted (1-6C)alkyl, (2-6C)alkenyl, or (2-6C)alkynyl group wherein the optional substituents are selected from cyano, halo, or a group of sub-formula:

—W—R⁹
wherein W is selected from —O—, —S(O)ₚ— (where p is 0, 1 or 2), —CO—, —NR^{e}CO—, —CONR^{e}, —NR^{e}CONR^{e}—, —SO₂NR^{e}—, —NR^{e}SO₂—, or —NR^{e}COO—;
R^{e} and R^{f} are independently selected from hydrogen or (1-2C)alkyl;
and R⁹ is selected from hydrogen or (1-4C)alkyl;
or —NR¹⁰R¹¹, where R¹⁰ and R¹¹ are independently selected from hydrogen, (1-2C)alkanoyl or (1-2C)alkyl, or R¹⁰ and R¹¹ are linked to form a 4, 5, 6 or 7 membered heterocyclic ring which optionally comprises, in addition to the nitrogen atom to which R¹⁰ and R¹¹ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
(iii) a group —NR¹²R¹³, wherein R¹² and R¹³ are each independently selected from hydrogen or (1-6C)alkyl, or R¹² and R¹³ are linked to form a 4, 5, 6 or 7-membered heterocyclic ring which optionally comprises, in addition to the nitrogen atom to which R¹² and R¹³ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl; or
(iv) a group of formula (II):

—X³—R¹⁴
wherein X³ is selected from —O—, —S(O)ₚ— (where p is 0, 1 or 2), —CO—, —NR^{g}CO—, —CONR^{g}—, —NR^{g}COO—, and —NR^{g}SO₂—,
where R^{g} is selected hydrogen or (1-2C)alkyl;
R¹⁴ is a (1-4C)alkyl group which is optionally substituted by halo, hydroxy, cyano, (1-4C)alkoxy, or R¹⁴ is

—NR¹⁵R¹⁶
where R¹⁵ and R¹⁶ are independently selected from hydrogen, (1-2C)alkanoyl or (1-2C)alkyl, or R¹⁵ and R¹⁶ are linked to form a 4, 5, 6 or 7-membered heterocyclic ring which optionally comprises, in addition to the nitrogen atom to which R¹⁵ and R¹⁶ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom is optionally substituted by (1-4C)alkyl, hydroxy(1-4C)alkyl, (1-2C)alkoxy-(1-4C)alkyl, or (1-4C)alkanoyl;
   R⁴ is a group —NR¹⁷R¹⁸, wherein R¹⁷ and R¹⁸ are linked to form a 4, 5, 6 or 7 membered heterocyclic ring which optionally comprises, in addition to the nitrogen atom to which R¹⁷ and R¹⁸ are attached, one or two further heteroatoms selected from O, N or S, and wherein any S atoms that are present may be optionally oxidised to form an SO or SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
   or a pharmaceutically acceptable salt thereof.

It is to be understood that, insofar as certain of the compounds of Formula I defined above may exist in optically active or racemic forms by virtue of one or more asymmetric carbon atoms, the invention includes in its definition any such optically active or racemic form which possesses the above-mentioned activity. The synthesis of optically active forms may be carried out by standard techniques of organic chemistry well known in the art, for example by synthesis from optically active starting materials or by the resolution of a racemic form. Similarly, the above-mentioned activity may be evaluated using the standard laboratory techniques referred to hereinafter.

It is to be understood that certain compounds of Formula I defined above may exhibit the phenomenon of tautomerism. In particular, tautomerism may affect any heterocyclic groups that bear 1 or 2 oxo substituents. It is also to be understood that the present invention includes in its definition any such tautomeric form, or a mixture thereof, which possesses the above-mentioned activity and is not to be limited merely to any one tautomeric form utilised within the formulae drawings or named in the Examples.

It is to be understood that certain compounds of Formula I above may exist in unsolvated forms as well as solvated forms, such as, for example, hydrated forms. It is also to be understood that the present invention encompasses all such solvated forms that possess anticancer or antitumour activity.

It is also to be understood that certain compounds of the Formula I may exhibit polymorphism, and that the present invention encompasses all such forms which possess anticancer or antitumour activity.

In this specification the generic term “alkyl” includes both straight-chain and branched-chain alkyl groups such as propyl, isopropyl and tert-butyl. However references to individual alkyl groups such as “propyl” are specific for the straight-chain version only, references to individual branched-chain alkyl groups such as “isopropyl” are specific for the branched-chain version only. An analogous convention applies to other generic terms, for example (1-4C)alkoxy includes methoxy, ethoxy and isopropoxy.

The term “halo” refers to fluoro, chloro, bromo, or iodo.

The term “heterocyclic ring”, unless otherwise defined herein, refers to saturated, partially saturated or unsaturated monocyclic rings containing 4, 5, 6 or 7 ring atoms. In particular compounds of the invention, “heterocyclic rings” are saturated monocyclic rings that contain 4, 5, 6 or 7 ring atoms, and especially 5 or 6 ring atoms.

Examples and suitable values of the term “heterocyclic ring” used herein are pyrrolidinyl, imidazolidinyl, pyrazolidinyl, piperidinyl, piperazinyl, morpholin-4-yl, thiomorpholin-4-yl, 1,4-oxazepan-4-yl, diazepanyl and oxazolidinyl.

Particular novel compounds of the invention include, for example, compounds of Formula I, or pharmaceutically-acceptable salts thereof, wherein, unless otherwise stated, each of R¹, R², R^{2a}, R³, R⁴, or Q has any of the meanings defined hereinbefore or in paragraphs (1) to (36) hereinafter:—
(1) R¹ is (1-4C)alkyl;
(2) R¹ is selected from methyl, ethyl, propyl, isopropyl, 2-methylpropyl or cyclopropylmethyl;
(3) R¹ is selected from methyl, ethyl, isopropyl or cyclopropylmethyl;
(4) R¹ is methyl;
(5) R¹ is isopropyl;
(6) R¹ is cyclopropylmethyl;
(7) R¹ is ethyl;
(8) Q is selected from a group (a), (b) or (c) as defined above, where A₂ is nitrogen, and the others are —CR^{2a},
(8) Q is selected from a group of formula (a) or (b) as defined above;
(9) Q is a group of formula (a) as defined above;
(10) Q is a group of formula (a) as defined above which is selected from

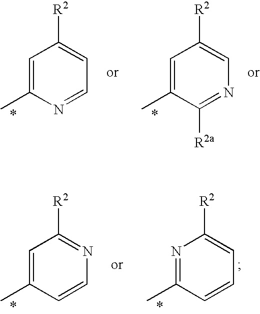

where R² and R^{2a} are as defined above;
(11) Q is a group of formula (b) as defined above;
(12) Q is a group of formula (b) as defined above which is selected from

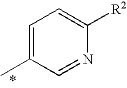

(13) R² is selected from (1-2C)alkyl, (1-2C)alkoxy, fluoro, chloro, cyano, hydroxy(1-2C)alkyl, or a group of sub-formula:

—X¹—R^{y}
where X¹ is selected from —NR^{a}—CO—, —S(O)_{z}— (where z is 0, 1 or 2); R^{a} is selected from hydrogen or methyl, and R^{y} is hydrogen or (1-2C)alkyl;
(14) R² group is selected from (1-2C)alkyl, (1-2C)alkoxy, fluoro, chloro, cyano, hydroxy(1-2C)alkyl, or a group of sub-formula:

—X¹—R^{y}
where X¹ is selected from —NR^{a}—CO—, —S(O)_{z}— (where z is 0, 1 or 2); R^{a} is selected from hydrogen or methyl, and R^{y} is hydrogen or (1-2C)alkyl;
(15) R² is selected from methyl, fluoro, chloro, hydroxymethyl, methoxy, acetamido, or methylthio;
(16) R² is selected from methyl, fluoro, chloro, hydroxymethyl, or methoxy;
(17) R² is selected from fluoro or chloro;
(18) R² is selected from methyl or hydroxymethyl;
(19) R² is methyl;
(20) R² is hydroxymethyl;
(21) R² is selected from acetamido or methoxy;
(22) R² is methoxy;
(23) each group R^{2a} present is independently selected from hydrogen, methyl, fluoro, chloro, hydroxymethyl, methoxy, acetamido, or methylthio;
(24) one R^{2a} present is selected from methyl, fluoro, chloro, hydroxymethyl, methoxy, acetamido, or methylthio, and the others are all hydrogen;
(25) one group R^{2a} present is selected from methoxy, methyl, fluoro, or chloro and the others are all hydrogen;
(26) each group R^{2a} present is hydrogen;
(27) R³ is selected from:
   (i) hydrogen, halo, nitro, cyano, or hydroxy;
   (ii) an optionally substituted (1-6C)alkyl group, wherein the optional substituents are selected from cyano, halo, or a group of sub-formula:

—W—R⁹
wherein W is selected from —O—, —S(O)ₚ— (where p is 0, 1 or 2), —CO—, —NR^{e}CO—, or —CONR^{e}—; R^{e} is selected from hydrogen or (1-2C)alkyl;
and R⁹ is selected from hydrogen or (1-4C)alkyl;
or —NR¹⁰R¹¹ where R¹⁰ and R¹¹ are independently selected from hydrogen, (1-2C)alkanoyl or (1-2C)alkyl, or R¹⁰ and R¹¹ are linked to form a 5, or 6 membered heterocyclic ring which optionally comprises, in addition to the nitrogen atom to which R¹⁰ and R¹¹ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom present in the ring is optionally substituted by (1-4C)alkyl or (1-4C)alkanoyl;
(iii) a group —NR¹²R¹³, wherein R¹² and R¹³ are each independently selected from hydrogen or (1-6C)alkyl, or R¹² and R¹³ are linked to form a 5, 6 or 7-membered heterocyclic ring which comprises, in addition to the nitrogen atom to which R¹² and R¹³ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom present in the ring is optionally substituted by (1-4C)alkyl or (1-4C)alkanoyl; or
(iv) a group of formula (II):

—X³—R¹⁴
wherein X³ is selected from —O—, —S(O)ₚ— (where p is 0, 1 or 2), —CO—, —NR^{g}CO—, —CONR^{g}—, or —NR^{g}COO—,
where R^{g} is selected hydrogen or (1-2C)alkyl;
R¹⁴ is a (1-4C)alkyl group which is optionally substituted by halo, hydroxy, cyano, (1-4C)alkoxy, or R¹⁴ is

—NR¹⁵R¹⁶
where R¹⁵ and R¹⁶ are independently selected from hydrogen, (1-2C)alkanoyl or (1-2C)alkyl, or R¹⁵ and R¹⁶ are linked to form a 5, or 6-membered heterocyclic ring which optionally comprises, in addition to the nitrogen atom to which R¹⁵ and R¹⁶ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom present in the ring is optionally substituted by (1-4C)alkyl or (1-4C)alkanoyl;
(28) R³ is selected from:
   (i) hydrogen, halo, cyano, or hydroxy;
   (ii) an optionally substituted (1-4C)alkyl group wherein the optional substituents are selected from cyano, halo, a group of sub-formula:

—W—R⁹
wherein W is selected from —O—, —S(O)ₚ— (where p is 0, 1 or 2), —CO—, —NR^{e}CO—, or —CONR^{e}—; R^{e} is selected from hydrogen or (1-2C)alkyl and R⁹ is selected from hydrogen or (1-4C)alkyl;
or —NR¹⁰R¹¹, where R¹⁰ and R¹¹ are independently selected from hydrogen or (1-2C)alkyl, or R¹⁰ and R¹¹ are linked to form a 5 or 6 membered heterocyclic ring which optionally comprises, in addition to the nitrogen atom to which R¹⁰ and R¹¹ are attached, one or two further heteroatoms selected from O, N or S, and wherein the ring is optionally substituted on any available carbon atom by one or two substituent groups selected from oxo, halo, hydroxy, cyano, or (1-4C)alkyl, and any available nitrogen atom present in the ring is optionally substituted by (1-4C)alkyl;
(iii) a group —NR¹²R¹³, wherein R¹² and R¹³ are each independently selected from hydrogen or (1-6C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, or (1-4C)alkyl, and any available nitrogen atom present in the ring is optionally substituted by (1-4C)alkyl; or
(iv) a group of formula (II):

—X³—R¹⁴
wherein X is selected from —O—, —S(O)ₚ— (where p is 0, 1 or 2), or —CONR^{g}—,
where R^{g} is selected hydrogen or (1-2C)alkyl;
R¹⁴ is a (1-4C)alkyl group which is optionally substituted by halo, hydroxy, cyano, (1-4C)alkoxy;
(29) R³ is selected from:
   (i) hydrogen, halo, or cyano;
   (ii) an optionally substituted (1-2C)alkyl group wherein the optional substituents are selected from cyano, halo, a group of sub-formula:

—W—R⁹
wherein W is selected from —O—, —S(O)ₚ— (where p is 0, 1 or 2), —CO—, —NR^{e}CO—, or —CONR^{e}—; R^{e} is selected from hydrogen or (1-2C)alkyl and R⁹ is selected from hydrogen or (1-4C)alkyl;
or —NR¹⁰R¹¹, where R¹⁰ and R¹¹ are independently selected from hydrogen or (1-2C)alkyl), or R¹⁰ and R¹¹ are linked to form a 5 or 6 membered heterocyclic ring which optionally comprises, in addition to the nitrogen atom to which R¹⁰ and R¹¹ are attached, one or two further heteroatoms selected from O, N or S, and wherein the ring is optionally substituted on any available carbon atom by one or two substituent groups selected from oxo, halo, hydroxy, cyano, or (1-4C)alkyl, and any available nitrogen atom present in the ring is optionally substituted by (1-4C)alkyl;
(iii) a group —NR¹²R¹³, wherein R¹² and R¹³ are each independently selected from hydrogen or (1-6C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, or (1-4C)alkyl, and any available nitrogen atom present in the ring is optionally substituted by (1-4C)alkyl; or
(iv) a group of formula (II):

—X³—R¹⁴
wherein X³ is selected from —O—, —S(O)ₚ— (where p is 0, 1 or 2), or —CONR^{g}—, where R^{g} is selected hydrogen or (1-2C)alkyl;
R¹⁴ is a (1-4C)alkyl group which is optionally substituted by halo, hydroxy, cyano, (1-4C)alkoxy;
(30) R³ is a group —NR¹²R¹³, wherein R¹² and R¹³ are each independently selected from hydrogen or (1-6C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom present in the ring is optionally substituted by (1-4C)alkyl or (1-4C)alkanoyl;
(31) R⁴ is a group —NR¹⁷R¹⁸, wherein R¹⁷ and R¹⁸ are linked to form a 5 or 6 membered heterocyclic ring which optionally comprises, in addition to the nitrogen atom to which R¹⁷ and R¹⁸ are attached, one or two further heteroatoms selected from O, N or S, and wherein any S atoms that are present may be optionally oxidised to form an SO or SO₂ group, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl, hydroxy(1-4C)alkyl, or (1-4C)alkanoyl;
(32) R⁴ is a group —NR¹⁷R¹⁸, wherein R¹⁷ and R¹⁸ are linked to form a 6 membered heterocyclic ring which optionally comprises, in addition to the nitrogen atom to which R¹⁷ and R¹⁸ are attached, one or two further heteroatoms selected from O, N or S, and wherein the ring is optionally substituted on any available carbon atom by one or two substituent groups selected from oxo, halo, hydroxy, cyano, or (1-4C)alkyl, and any available nitrogen atom is optionally substituted by (1-4C)alkyl, hydroxy(1-4C)alkyl or (1-4C)alkanoyl;
(33) R⁴ is a group of formula:

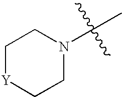

wherein Y is selected from O, S, NR²⁰, or CR²¹, where R²⁰ is selected from hydrogen, (1-2C)alkyl, hydroxy(1-2C)alkyl, (1-2C)alkoxy(1-2C)alkyl, or (1-2C)alkanoyl, and R²¹ is selected from hydrogen, hydroxy, (1-2C)alkyl, hydroxy(1-2C)alkyl, (1-2C)alkoxy(1-2C)alkyl, or (1-2C)alkanoyl;
(34) R⁴ is a group of formula:

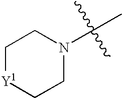

wherein Y¹ is selected from O, NR²², or CR²³, where R²² is selected from hydrogen or (1-2C)alkyl, and R²³ is selected from hydrogen or hydroxy;
(35) R⁴ is selected from morpholin-4yl, 4-methylpiperazin-1-yl, or 4-hydroxypiperidin-1-yl;
(36) R⁴ is morpholin-4-yl.

In a particular group of compounds of the Formula I, R¹ is an alkyl group as defined in any one of paragraphs (1) to (7) above. In a further group of compounds of the invention, R¹ is methyl.

In a further group of compounds of Formula I, R² is as defined in paragraphs (13) to (22) above and each R^{2a} group that may be present is as defined in any one of paragraphs (23) to (26) above. In a particular group of compounds of the invention, each R² group present is as defined in any one of paragraphs (15) to (22) above.

In a further group of compounds, the group —NR¹Q in the 4-position of the pyrimidine ring has the following structure:

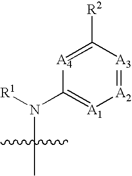

wherein R¹ and R² have any one of the definitions set out herein, and one of A₁, A₂, A₃ and A₄ is nitrogen and the others are —CR^{2a}, and in particular, all or all except one R^{2a} groups are hydrogen.

In a further group of compounds, the group —NR¹Q in the 4-position of the pyrimidine ring has the following structure:

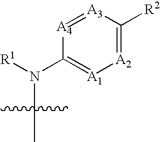

wherein R¹ and R² have any one of the definitions set out herein, and one of A₁, A₂, A₃ and A₄ is nitrogen and the others are —CR^{2a} where R^{2a} has any of the definitions set out above and in particular, all or all except one R^{2a} groups are hydrogen. In one particular embodiment, all R^{2a} groups are hydrogen. In another embodiment, one R^{2a} group is other than hydrogen, and in particular is methoxy, methyl, fluoro, or chloro, and the remainder are hydrogen. Where one of R^{2a} is other than hydrogen, it is suitably arranged in a position on the ring Q which is meta or para to the R² group.

In a further sub-group of compounds, the R² group present is methoxy or chloro.

In a further particular group of compounds of the invention, R³ is as defined in any one of paragraphs (27) to (30) above, and is especially as defined in paragraphs (29) or (30) above.

In a particular group of compounds of the invention, R⁴ is as defined in any one of paragraphs (31) to (36) above. In a further particular group of compounds of the invention, R⁴ is as defined in either paragraph (35) or (36). Suitably, R⁴ is morpholin-4yl.

In a group of compounds of formula I, Q, R¹ and R³ have any one of the definitions set out hereinbefore, R⁴ is a group of formula:

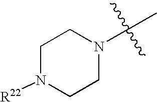

wherein R²² is selected from hydrogen or (1-2C)alkyl.

In a sub-group of compounds of Formula I, Q, R¹ and R³ have any one of the definitions set out hereinbefore, and R⁴ is a group of formula:

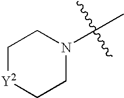

wherein Y² is O or —CR²³, and R²³ is selected from hydrogen or hydroxyl.

A particular sub-group of compounds of the invention have the structural formula IA:

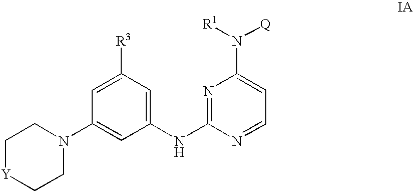

wherein:
Y is selected from O, S, NR²⁰, or CR²¹, where R²⁰ is selected from hydrogen, (1-2C)alkyl, hydroxy(1-2C)alkyl, (1-2C)alkoxy(1-2C)alkyl, or (1-2C)alkanoyl, and R²¹ is selected from hydrogen, hydroxy, (1-2C)alkyl, hydroxy(1-2C)alkyl, (1-2C)alkoxy(1-2C)alkyl, or (1-2C)alkanoyl;
R¹ is a (1-4C)alkyl group;
Q is selected from a group of formula:

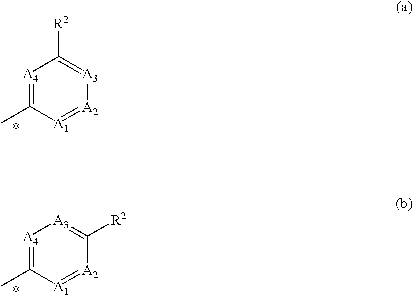

wherein * is the point of attachment to the compound of formula I above;
one of A₁, A₂, A₃, and A₄ is N and the others are —CR^{2a}—;
R² is independently selected from (1-2C)alkyl, (1-2C)alkoxy, fluoro, chloro, cyano, hydroxy(1-2C)alkyl, or a group of sub-formula:

—X¹—R^{y}

where X¹ is selected from —CO—, —NR^{a}—, —NR^{a}—CO—, —NR^{a}—COO—, NR^{a}CONR^{b}, —CONR^{a}—, —S(O)_{z}— (where z is 0, 1 or 2); —SO₂NR^{a}—, and —NR^{a}SO₂—, R^{a} and R^{b} are each independently selected from hydrogen or methyl, and R^{y} is hydrogen or (1-2C)alkyl;
each R^{2a} group present is independently selected from hydrogen, (1-2C)alkyl, (1-2C)alkoxy, fluoro, chloro, cyano, hydroxy(1-2C)alkyl, or a group of sub-formula:

—X²—R^{z}

where X² is selected from —CO—, —NR^{c}—, —NR^{c}—CO—, —CONR^{c}—, —S(O)_{z}— (where z is 0, 1 or 2), R^{c} is selected from hydrogen or methyl, and R^{z} is hydrogen or (1-2C)alkyl;
R³ is selected from:
(i) hydrogen, halo, nitro, cyano, or hydroxy;
(ii) an optionally substituted (1-4C)alkyl group wherein the optional substituents are selected from cyano, halo, or a group of sub-formula:

—W—R⁹
wherein W is selected from —O—, —S(O)ₚ— (where p is 0, 1 or 2), —CO—, —NR^{e}CO—, —CONR^{e}—;
R^{e} is selected from hydrogen or (1-2C)alkyl
and R⁹ is selected from hydrogen or (1-2C)alkyl;
or —NR¹⁰R¹¹, where R¹⁰ and R¹¹ are independently selected from hydrogen or (1-2C)alkyl, or R¹⁰ and R¹¹ are linked to form a 5, or 6 membered heterocyclic ring which optionally comprises, in addition to the nitrogen atom to which R¹⁰ and R¹¹ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl;
(iii) a group —NR¹²R¹³, wherein R¹² and R¹³ are each independently selected from hydrogen or (1-2C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl; or
(iv) a group of formula (II):

—X³—R¹⁴
wherein X³ is selected from —O—, —S(O)ₚ— (where p is 0, 1 or 2), —CO—, —NR^{g}CO—, or —CONR^{g}—,
R^{g} is selected hydrogen or (1-2C)alkyl, and
R¹⁴ is a (1-4C)alkyl group which is optionally substituted by halo, hydroxy, cyano, (1-4C)alkoxy, or R¹⁴ is

NR¹⁵R¹⁶
where R¹⁵ and R¹⁶ are independently selected from hydrogen or (1-2C)alkyl, or R¹⁵ and R¹⁶ are linked to form a 5, or 6-membered heterocyclic ring which optionally comprises, in addition to the nitrogen atom to which R¹⁵ and R¹⁶ are attached, one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl;
   or a pharmaceutically acceptable salt thereof.

In a particular group of compounds of Formula IA, Y is selected from O, NR²⁰ or CR²¹, where R²⁰ is selected from hydrogen or (1-2C)alkyl, and R²¹ is selected from hydrogen or hydroxy. In a further group of compounds of Formula IA, Y is selected from O or NR²⁰, where R²⁰ is selected from hydrogen or (1-2C)alkyl. In a further group of compounds of Formula IA, Y is O.

In compounds of Formula IA, R¹ is suitably has any one of the definitions set out in paragraphs (2) to (7) above. In a particular group of compounds of Formula IA, R¹ is methyl.

In a particular group of compounds of Formula IA, R² has any one of the definitions set out herein before or has any one of the definitions set out in paragraphs (13) to (22) above, and each R^{2a} group has any one of the definitions set out in paragraphs (27) to (30) above.

In a particular group of compounds of Formula IA, R³ is as defined in either of paragraphs (29) or (30 above.

A further sub-group of compounds of the invention have the structural formula IB shown below

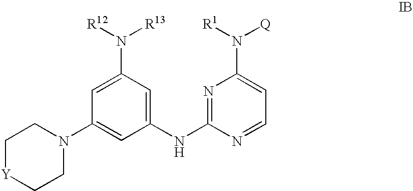

wherein:

Y, R¹ and Q each have any one of the definitions set out above in relation to Formula IA;

R¹² and R¹³ are each independently selected from hydrogen or (1-6C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl;

or a pharmaceutically acceptable salt thereof.

In compounds of Formula IB, R¹² and R¹³ are suitably linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl.

In a further sub-group of compounds of Formula IB, R¹² and R¹³ are linked to form a 5, 6 or 7-membered heterocyclic ring, and wherein, in addition to the nitrogen atom to which R¹² and R¹³ are attached, the ring optionally comprises one further heteroatom selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom is optionally substituted by (1-4C)alkyl or (1-4C)alkanoyl.

A further particular sub-group of compounds of Formula I have the structural formula IC shown below

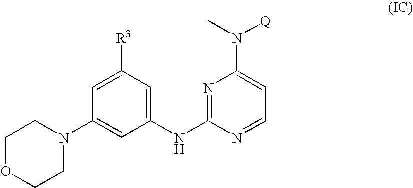

wherein Q and R³ have any one of the definitions set out above in relation to Formula I, or a pharmaceutically acceptable salt thereof.

A further particular sub-group of compounds of Formula I have the structural formula ID shown below

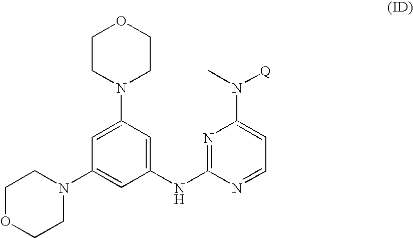

wherein Q has any one of the definitions set out above in relation to Formula I,
or a pharmaceutically acceptable salt thereof.

Particular novel compounds of the invention include any one of the following:
N-(3,5-dimorpholin-4-ylphenyl)-N′-(4-methoxypyridin-2-yl)-N′-methyl-pyrimidine-2,4-diamine;
N′-(4-chloropyridin-2-yl)-N-(3,5-dimorpholin-4-ylphenyl)-N′-methyl-pyrimidine-2,4-diamine;
N′-(2-chloropyridin-4-yl)-N-(3,5-dimorpholin-4-ylphenyl)-N′-methyl-pyrimidine-2,4-diamine;
N′-(5-chloropyridin-3-yl)-N-(3,5-dimorpholin-4-ylphenyl)-N′-methyl-pyrimidine-2,4-diamine;
N′-(6-chloropyridin-2-yl)-N-(3,5-dimorpholin-4-ylphenyl)-N′-methyl-pyrimidine-2,4-diamine;
N-(3,5-dimorpholin-4-ylphenyl)-N′-(6-methoxypyridin-2-yl)-N′-methyl-pyrimidine-2,4-diamine;

N4-(6-chloropyridin-3-yl)-N2-(3,5-dimorpholinophenyl)-N4-methylpyrimidine-2,4-diamine;
N-(3,5-dimorpholin-4-ylphenyl)-N′-methyl-N′-(6-methylpyridin-3-yl)pyrimidine-2,4-diamine;
N-(3,5-dimorpholin-4-ylphenyl)-N′-(5-methoxypyridin-3-yl)-N′-methyl-pyrimidine-2,4-diamine;
N′-(2,5-dimethylpyridin-3-yl)-N-(3,5-dimorpholin-4-ylphenyl)-N′-methyl-pyrimidine-2,4-diamine;
N-(3,5-dimorpholin-4-ylphenyl)-N′-(5-methoxy-2-methyl-pyridin-3-yl)-N′-methyl-pyrimidine-2,4-diamine;
N′-(6-chloro-5-methoxy-pyridin-3-yl)-N-(3,5-dimorpholin-4-ylphenyl)-N′-methyl-pyrimidine-2,4-diamine;
N′-(6-chloro-5-methyl-pyridin-3-yl)-N-(3,5-dimorpholin-4-ylphenyl)-N′-methyl-pyrimidine-2,4-diamine;
N′-(2-chloro-5-methoxy-pyridin-3-yl)-N-(3,5-dimorpholin-4-ylphenyl)-N′-methyl-pyrimidine-2,4-diamine;
(6-((2-(3,5-dimorpholinophenylamino)pyrimidin-4-yl)(methyl)amino)-5-methylpyridin-2-yl)methanol;
N-[3,5-di(morpholin-4-yl)phenyl]-N′-methyl-N′-(6-methylpyridin-2-yl)pyrimidine-2,4-diamine;
N-[3,5-di(morpholin-4-yl)phenyl]-N′-methyl-N′-(5-methylpyridin-2-yl)pyrimidine-2,4-diamine; or
[5-[[2-[[3,5-di(morpholin-4-yl)phenyl]amino]pyrimidin-4-yl]-methylamino]-6-methylpyridin-3-yl]methanol;
   or a pharmaceutically acceptable salt thereof

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

The compounds of the invention may be administered in the form of a pro-drug that is a compound that is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula I, IA, IB, IC or ID, and in vivo cleavable amide derivatives that may be formed at a carboxy group or an amino group in a compound of the Formula I, IA, IB, IC or ID.

Accordingly, the present invention includes those compounds of the Formula I, IA, IB, IC or ID as defined hereinbefore when made available by organic synthesis and when made available within the human or animal body by way of cleavage of a pro-drug thereof. Accordingly, the present invention includes those compounds of the Formula I, IA, IB, IC or ID that are produced by organic synthetic means and also such compounds that are produced in the human or animal body by way of metabolism of a precursor compound, that is a compound of the Formula I, IA, IB, IC or ID may be a synthetically-produced compound or a metabolically-produced compound.

A suitable pharmaceutically-acceptable pro-drug of a compound of the Formula I, IA, IB, IC or ID is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the Formula I, IA, IB, IC or ID that possesses a carboxy group is, for example, an in vivo cleavable ester thereof. An in vivo cleavable ester of a compound of the Formula I containing a carboxy group is, for example, a pharmaceutically-acceptable ester, which is cleaved in the human or animal body to produce the parent acid. Suitable pharmaceutically-acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically-acceptable pro-drug of a compound of the Formula I, IA, IB, IC or ID that possesses a hydroxy group is, for example, an in vivo cleavable ester or ether thereof. An in vivo cleavable ester or ether of a compound of the Formula I, IA, IB, IC or ID containing a hydroxy group is, for example, a pharmaceutically-acceptable ester or ether, which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-[di-(1-4C)alkyl]carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically-acceptable ether forming groups for a hydroxy group include α-acyloxyalkyl groups such as acetoxymethyl and pivaloyloxymethyl groups.

A suitable pharmaceutically-acceptable pro-drug of a compound of the Formula I, IA, IB, IC or ID that possesses an amino group is, for example, an in vivo cleavable amide derivative thereof. Suitable pharmaceutically-acceptable amides from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl.

The in vivo effects of a compound of the Formula I, IA, IB, IC or ID may be exerted in part by one or more metabolites that are formed within the human or animal body after administration of a compound of the Formula I, IA, IB, IC or ID. As stated hereinbefore, the in vivo effects of a compound of the Formula I, IA, IB, IC or ID may also be exerted by way of metabolism of a precursor compound (a pro-drug).

According to a further aspect of the invention there is provided a pharmaceutical composition, which comprises a compound of the formula I, or a pharmaceutically acceptable salt thereof, as defined hereinbefore in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The compound of formula I will normally be administered to a warm-blooded animal at a unit dose within the range 5-5000 mg/m² body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the practitioner who is treating any particular patient may determine the optimum dosage.

### Preparation of Compounds of Formula I

It will be appreciated by a person skilled in the art that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Furthermore, the synthesis of optically active forms may be carried out by standard techniques of organic chemistry well known in the art, for example by synthesis from optically active starting materials or by resolution of a racemic form.

Compounds of formula I can be prepared by various conventional methods as would be apparent to a chemist. In particular, compounds of formula I may be prepared by reacting a compound of formula (II):

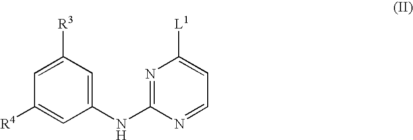

where R³ and R⁴ is as defined in relation to formula I with the proviso that any functional groups are optionally protected, and L¹ is a leaving group, with a compound of formula (III)

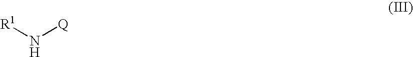

where R¹ and Q are as defined in relation to formula I provided that any functional groups are optionally protected. Thereafter, any protecting groups can be removed using conventional methods, and if required, the compound of formula I can be converted to a different compound of formula I or a salt, again using conventional chemical methods well known in the art.

Suitable leaving groups L are halogeno such as chloro. The reaction is suitably carried out in an organic solvent such as a C₁₋₆alkanol, for instance, n-butanol, isopropanol or 2-pentanol, dimethylacetamide (DMA), or N-methylpyrrolidine (NMP) or mixtures thereof. An acid, and in particular an inorganic acid such as hydrochloric acid, is suitably added to the reaction mixture. The reaction is suitably conducted at elevated temperatures for example at from 80-150° C., conveniently at the reflux temperature of the solvent.

Alternatively, the reaction between (II) and (III) may be catalysed by transition metals complexes, such as palladium catalysts. Examples of suitable palladium catalysts include Pd2(dba)3 (tris(dibenzylideneacetone)dipalladium), Pd(PPh₃)₄ and Pd(OAc)₂. This palladium catalysed reaction conveniently carried out in the presence of a suitable base, such as potassium carbonate, cesium carbonate, potassium phosphate, sodium tert-butoxide, or 1,8-diazabicyclo[5.4.0]undec-7-ene (DBU). Suitable solvents for such a reaction include toluene, dioxane or ethylene glycol dimethylether (DME). Suitable ligands for use in such a reaction include Xantphos (4,5-bis(diphenylphosphino)-9,9-dimethylxanthene), BINAP (2,2′-bis(diphenylphosphino)-1,1′-binaphtyl) or DPPF (1,1′-bis(diphenylphosphino)ferrocene). The reaction is conveniently carried out at an elevated temperature, generally at the reflux temperature of the particular solvent used. A temperature of 90-140° C. would be typical.

Compounds of formula (II) may be prepared by various methods including for example, where L is a halogen, by reacting a compound of formula (IV)

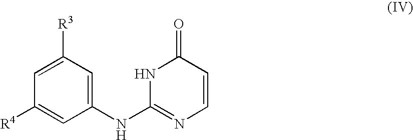

where R⁴ is as defined in relation to formula I, with a suitable halogenating agent such as phosphorus oxychloride.

The reaction is conducted under reactions conditions appropriate to the halogenating agent employed. For instance, it may be conducted at elevated temperatures, for example of from 50-100° C., in an organic solvent such as acetonitrile or dichloromethane (DCM).

Compounds of formula (IV) are suitably prepared by reacting a compound of formula (V)

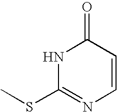

with a compound of formula (VI)

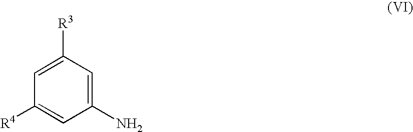

where R³ and R⁴ are as defined in relation to formula I.

The reaction is suitably effected in an organic solvent such as diglyme, again at elevated temperatures, for example from 120-180° C., and conveniently at the reflux temperature of the solvent.

Compounds of formula (II), in which L¹ is chloro, may also be prepared by reacting a compound of formula XIII

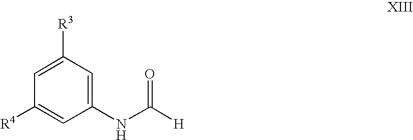

wherein R³ and R⁴ are as defined in relation to Formula I with 4-chloro-2-methylsulfonylpyrimidine in the presence of a suitable base, such as sodium hydride.

Alternatively, compounds of formula I may be prepared by reaction a compound of formula (VII)

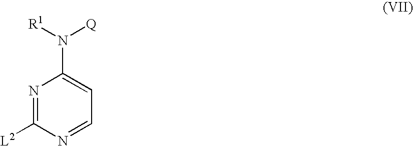

where R¹ and Q are as defined in relation to formula I provided that any functional groups can be optionally protected, and L² is a leaving group similar to those defined in relation to formula (II) or may be —SO₂Me, with a compound of formula (VI) as defined above.

Again, any protecting groups can be removed using conventional methods, and if required, the compound of formula I can be converted to a different compound of formula I or a salt, again using conventional chemical methods.

Conditions for carrying out such a reaction are broadly similar to those required for the reaction between compounds (II) and (III) described above.

Compounds of formula (VII) are suitably prepared by reacting a compound of formula (III) as defined above with a compound of formula (VIII)

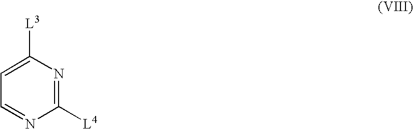

where L³ and L⁴ are leaving groups such as halogen, and in particular chloro.

The reaction is suitably effected in the presence of an organic base such as triethylamine. The reaction is also suitably carried out at an elevated temperature, for example between 80 and 120° C. in a suitable organic solvent such as a C₁₋₆alkanol, for instance, ethanol. The reaction can also be performed in presence of a strong base such as sodium hydride, LIHMDS or NaHMDS, in an organic solvent such as DMA or THF. When the basic reaction conditions are used, depressed temperatures, for example from −20° C. to 20° C., conveniently at about 0° C. are suitably employed.

Compounds of formula (VII) can also be prepared by reacting a compound of formula (IX)

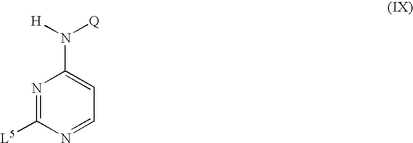

wherein L⁵ is a leaving group as defined hereinbefore and Q is as defined in relation to Formula I with a compound

R¹—X

where X is a suitable leaving group such as halogen and R¹ is as defined above in relation to Formula I.

This reaction is conveniently performed using a base such as caesium carbonate in a suitable solvent, such as, for example, dimethylformamide.

Another method to prepare compounds of formula I involves the reaction of a compound formula (X)

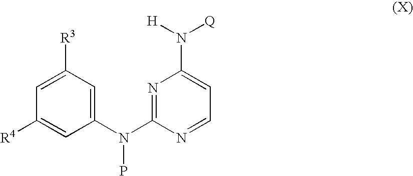

wherein Q, R³ and R⁴ are as defined above in relation to Formula I and P is a suitable protecting group for this reaction, for example a 4-methoxybenzyl group;

with a compound

R¹-L⁶

where L⁶ is a suitable leaving group such as halogen and R¹ is as defined above in relation to Formula I.

This reaction is conveniently performed using a strong base such as sodium hydride in a suitable solvent, for example dimethylformamide.

Another method to prepare compounds of formula I is to react a compound of formula (XI)

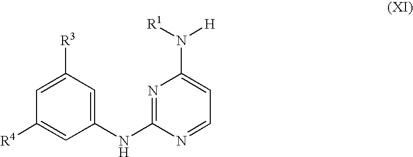

wherein R¹, R³ and R⁴ are as defined above in relation to Formula I;

with a compound of formula (XII)

L⁶-Q (XII)

wherein Q is as defined above in relation to Formula I and L⁶ is halogen, for example bromo.

This reaction is suitably carried out in the presence of a suitable catalyst such as a palladium catalyst. Examples of suitable palladium catalysts include Pd2(dba)3 (tris(dibenzylideneacetone)dipalladium), Pd(PPh₃)₄ and Pd(OAc)₂. This palladium catalysed reaction conveniently carried out in the presence of a suitable base, such as potassium carbonate, cesium carbonate, potassium phosphate, sodium tert-butoxide, or 1,8-diazabicyclo[5.4.0]undec-7-ene (DBU). Suitable solvents for such a reaction include toluene, dioxane or ethylene glycol dimethylether (DME). Suitable ligands for use in such a reaction include Xantphos (4,5-bis(diphenylphosphino)-9,9-dimethylxanthene), BINAP (2,2′-bis(diphenylphosphino)-1,1′-binapthyl) or DPPF (1,1′-bis(diphenylphosphino)ferrocene). The reaction is conveniently carried out at an elevated temperature, generally at the reflux temperature of the particular solvent used. A temperature of 90-140° C. would be typical.

Compounds of formula (III) are either known compounds or they can be prepared from known compounds using analogous methods, which would be apparent to the skilled chemist.

Compounds of the formula I can be converted into further compounds of the formula I using standard procedures conventional in the art. Examples of the types of conversion reactions that may be used to convert a compound of formula I to a different compound of formula I include introduction of a substituent by means of an aromatic substitution reaction or of a nucleophilic substitution reaction, reduction of substituents, alkylation of substituents and oxidation of substituents. The reagents and reaction conditions for such procedures are well known in the chemical art.

Particular examples of aromatic substitution reactions include the introduction of an alkyl group using an alkyl halide and Lewis acid (such as aluminium trichloride) under Friedel Crafts conditions; and the introduction of a halogeno group. Particular examples of nucleophilic substitution reactions include the introduction of an alkoxy group or of a monoalkylamino group, a dialkyamino group or a N-containing heterocycle using standard conditions. Particular examples of reduction reactions include the reduction of a carbonyl group to a hydroxy group with sodium borohydride or of a nitro group to an amino group by catalytic hydrogenation with a nickel catalyst or by treatment with iron in the presence of hydrochloric acid with heating.

The preparation of particular compounds of formula I, such as compounds of formula I, IA, IB, IC or ID using the above-described methods form a further aspect of the invention.

### Biological Assays

### A) In Vitro EphB4 Enzyme Assay

This assay detects inhibitors of EphB4-mediated phosphorylation of a polypeptide substrate using Alphascreen™ luminescence detection technology. Briefly, recombinant EphB4 was incubated with a biotinylated-polypeptide substrate (biotin-poly-GAT) in presence of magnesium-ATP. The reaction was stopped by addition of EDTA, together with streptavidin-coated donor beads which bind the biotin-substrate containing any phosphorylated tyrosine residues. Anti-phosphotyrosine antibodies present on acceptor beads bind to phosphorylated substrate, thus bringing the donor & acceptor beads into close proximity. Subsequent excitation of the donor beads at 680 nm generated singlet oxygen species that interact with a chemiluminescer on the acceptor beads, leading to light emission at 520-620 nm. The signal intensity is directly proportional to the level of substrate phosphorylation and thus inhibition is measured by a decrease in signal.

### Aqueous Compound Preparation:

Test compounds were prepared as 10 mM stock solutions in DMSO (Sigma-Aldrich Company Ltd, Gillingham, Dorset SP8 4XT Catalogue No. 154938) and serially diluted with 5% DMSO to give a range of test concentrations at 6× the required final concentration. A 2 μl aliquot of each compound dilution was transferred to appropriate wells of low volume white 384-well assay plates (Greiner, Stroudwater Business Park, Stonehouse, Gloucestershire, GL10 3SX, Cat No. 784075) in duplicate. Each plate also contained control wells: maximum signal was created using wells containing 2 μl of 5% DMSO, and minimum signal corresponding to 100% inhibition were created using wells containing 2 μl of 0.5M EDTA (Sigma-Aldrich Company Ltd, Catalogue No. E7889).

### Acoustic Compound Preparation:

Test compounds were prepared in 100% DMSO and dispensed in multiples of 2.5 nl droplets into the target wells of the assay plate using a Labcyte Echo550 (Sunnyvale, Calif. 94089, USA). To ensure that each well contained a total of 120 nl DMSO the wells were all backfilled as required. Maximum control wells contained DMSO, minimum control wells contained 120 nl of a compound at a concentration sufficient to completely inhibit enzyme activity. The test range of compounds was 100× the required final concentration.

For the assay using aqueous prepared compounds, in addition to the compound or control, each well of the assay plate contained; 10 μl of assay mix containing final buffer (10 mM Tris, 100 μM EGTA, 10 mM magnesium acetate, 4 μM ATP, 500 μM DTT, 1 mg/ml BSA), 0.25 ng of recombinant active EphB4 (amino acids 563-987; Swiss-Prot Acc. No. P54760) (ProQinase GmbH, Breisacher Str. 117, D-79106 Freiburg, Germany, Catalogue No 0178-0000-3) and 5 nM of the poly-GAT substrate (CisBio International, BP 84175, 30204 Bagnols/Cèze Cedex, France, Catalogue No. 61GATBLB). Assay plates were then incubated at room temperature for 1 hour.

For assays using compounds prepared via acoustic dispensing, the assay mix was adjusted such that the final assay volume of 12 ul contained the same concentration of reagent as 10 ul of assay mix used when aqueous compounds were tested.

Regardless of the method of compound preparation, the reaction was stopped by addition of 5 μl/well stop buffer (10 mM Tris, 495 mM EDTA, 1 mg/ml BSA) containing 0.25 ng each of AlphaScreen anti-phosphoTyrosine-100 acceptor beads and streptavidin-coated donor beads (Perkin Elmer, Catalogue No 6760620M). The plates were sealed under natural lighting conditions, wrapped in aluminium foil and incubated in the dark for a further 20 hours.

The resulting assay signal was determined on the Perkin Elmer EnVision plate reader. The minimum value was subtracted from all values, and the signal plotted against compound concentration to generate IC₅₀ data. The method used to generate the compound dilutions was recorded with the IC₅₀ value in the database. Data from compounds prepared using acoustic dispensing were marked “Echo” and the remaining results were marked “Genesis”. Compounds of the invention were tested in the in vitro EphB4 enzyme assay and the IC₅₀ values so obtained are presented in Table A below.

TABLE A

| Example | EphB4 enzyme assay | Method of compound |
|---|---|---|
| Number | Mean IC₅₀ value (μM) | preparation |
| 1 | 0.112 | Genesis |
| | 0.01362 | Echo |
| 2a | 0.405 | Genesis |
| 2b | 0.207* | Genesis |
| 3 | 0.049 | Genesis |
| | 0.01164 | Echo |
| 4 | 0.611 | Genesis |
| 5 | 0.087 | Genesis |
| | 0.00633 | Echo |
| 6 | 0.201* | Genesis |
| 7a | 0.116* | Genesis |
| 7b | 0.152* | Genesis |
| | 0.00358 | Echo |
| 7c | 0.384* | Genesis |
| 7d | 0.00437* | Echo |
| 8a | 0.106* | Genesis |
| 8b | 0.166* | Genesis |
| 9 | 0.0107* | Echo |
| 10 | 0.141* | Echo |
| 11 | >14.4* | Genesis |
| | 0.207* | Echo |
| 12 | 0.250* | Genesis |
| | 0.158* | Echo |
| 13 | 0.0669* | Echo |

*Tested once only.

### B) In Vitro EphB4 Cell Assay

The assay identifies inhibitors of cellular EphB4 by measuring a decrease in phosphorylation of EphB4 following treatment of cells with compound. The endpoint assay used a sandwich ELISA to detect EphB4 phosphorylation status. Briefly, Myc-tagged EphB4 from treated cell lysate was captured on the ELISA plate via an anti-c-Myc antibody. The phosphorylation status of captured EphB4 was then measured using a generic phosphotyrosine antibody conjugated to HRP via a colourimetric output catalysed by HRP, with level of EphB4 phosphorylation directly proportional to the colour intensity. Absorbance was measured spectrophotometrically at 450 nm.

Full length human EphB4 (Swiss-Prot Acc. No. P54760) was cloned using standard techniques from cDNA prepared from HUVEC using RT-PCR. The cDNA fragment was then sub-cloned into a pcDNA3.1 expression vector containing a Myc-His epitope tag to generate full-length EphB4 containing a Myc-His tag at the C-terminus (Invitrogen Ltd. Paisley, UK). CHO-K1 cells (LGC Promochem, Teddington, Middlesex, UK, Catalogue No. CCL-61) were maintained in HAM's F12 medium (Sigma-Aldrich Company Ltd, Gillingham, Dorset SP8 4XT, Catalogue No. N4888) containing 10% heat-inactivated foetal calf serum (PAA lab GmbH, Pasching, Austria Catalogue No. PAA-A15-043) and 1% glutamax-1 (Invitrogen Ltd., Catalogue No. 35050-038) at 37° C. with 5% CO₂. CHO-K1 cells were engineered to stably express the EphB4-Myc-His construct using standard stable transfection techniques, to generate cells hereafter termed EphB4-CHO.

For each assay, 10,000 EphB4-CHO cells were seeded into each well of Costar 96-well tissue-culture plate (Fisher Scientific UK, Loughborough, Leicestershire, UK., Catalogue No. 3598) and cultured overnight in full media. On day 2, the cells were incubated overnight in 90 μl/well of media containing 0.1% Hyclone stripped-serum (Fisher Scientific UK, Catalogue No. SH30068.02). Test compounds were prepared as 10 mM stock solutions in DMSO (Sigma-Aldrich Company Ltd, Gillingham, Dorset SP8 4XT Catalogue No. 154938) and serially diluted with serum-free media to give a range of test concentrations at 10× the required final concentration. A 10 μl aliquot of each compound dilution was transferred to the cell plates in duplicate wells, and the cells incubated for 1 hour at 37° C. Each plate also contained control wells: a maximum signal was created using untreated cells, and minimum signal corresponding to 100% inhibition was created using wells containing a reference compound known to abolish EphB4 activity.

Recombinant ephrin-B2-Fc (R&D Systems, Abingdon Science Park, Abingdon, Oxon OX14 3NB UK, Catalogue No. 496-EB), a Fc-tagged form of the cognate ligand for EphB4, was pre-clustered at a concentration of 3 μg/ml with 0.3 μg/ml anti-human IgG, Fc fragment specific (Jackson ImmunoResearch Labs, Northfield Business Park, Soham, Cambridgeshire, UK CB7 5UE, Catalogue No. 109-005-008) in serum-free media for 30 minutes at 4° C. with occasional mixing. Following compound treatment, cells were stimulated with clustered ephrin-B2 at a final concentration of 1 μg/ml for 20 minutes at 37° C. to induce EphB4 phosphorylation. Following stimulation, the medium was removed and the cells lysed in 100 μl/well of lysis buffer (25 mM Tris HCl, 3 mM EDTA, 3 mM EGTA, 50 mM NaF, 2 mM orthovanadate, 0.27M Sucrose, 10 mM β-glycerophosphate, 5 mM sodium pyrophosphate, 2% Triton X-100, pH 7.4).

Each well of an ELISA Maxisorp 96-well plate (Nunc; Fisher Scientific UK, Loughborough, Leicestershire, UK., Catalogue No. 456537) was coated overnight at 4° C. with 100 μl of anti-c-Myc antibody in Phosphate Buffered Saline (10 μg/ml; produced at AstraZeneca). Plates were washed twice with PBS containing 0.05% Tween-20 and blocked with 250 μl/well 3% TopBlock (Fluka) (Sigma-Aldrich Company Ltd, Gillingham, Dorset SP8 4XT, Catalogue No. 37766) for a minimum of 2 hours at room temperature. Plates were washed twice with PBS/0.05% Tween-20 and incubated with 100 μl/well cell lysate overnight at 4° C. ELISA plates were washed four times with PBS/0.05% Tween-20 and incubated for 1 hour at room temperature with 100 μl/well HRP-conjugated 4G10 anti-phosphotyrosine antibody (Upstate, Dundee Technology Park, Dundee, UK, DD2 1SW, Catalogue No. 16-105) diluted 1:6000 in 3% Top Block. ELISA plates were washed four times with PBS/0.05% Tween-20 and developed with 100 μl/well TMB substrate (Sigma-Aldrich Company Ltd, Catalogue No. T0440). The reaction was stopped after 15 minutes with the addition of 25 μl/well 2M sulphuric acid. The absorbances were determined at 450 nm using the Tecan SpectraFluor Plus. The minimum value was subtracted from all values, and the signal plotted against compound concentration to generate IC₅₀ data.

Compounds of the invention were active in the above assays showing IC₅₀ values of less than 1 μM, in Assay A and less than 3 μM in Assay B. For instance the Compound of Example 2a above showed an IC₅₀ of 0.405 μM in assay A and IC₅₀ of 0.197 μM in assay B. Preferred compounds of the invention show IC₅₀ values of less than 1 μM in both Assay A and Assay B.

As a result of their activity in screens described above, the compounds of the present invention are expected to be useful in the treatment of diseases or medical conditions mediated alone or in part by EphB4 enzyme activity, i.e. the compounds may be used to produce an EphB4 inhibitory effect in a warm-blooded animal in need of such treatment. Thus, the compounds of the present invention provide a method for treating the proliferation of malignant cells characterised by inhibition of the EphB4 enzyme, i.e. the compounds may be used to produce an anti-proliferative effect mediated alone or in part by the inhibition of EphB4.

According to another aspect of the present invention there is provided a compound of the formula I, IA, IB, IC or ID, or a pharmaceutically acceptable salt thereof, as defined hereinbefore for use in a method of treatment of the human or animal body by therapy.

Thus according to a further aspect of the invention there is provided a compound of the formula I, IA, IB, IC or ID, or a pharmaceutically acceptable salt thereof, as defined hereinbefore for use as a medicament.

According to a further aspect of the invention there is provided the use of a compound of the formula I, IA, IB, IC or ID, or a pharmaceutically acceptable salt thereof, as defined hereinbefore in the manufacture of a medicament for use in the production of an EphB4 inhibitory effect in a warm-blooded animal such as man.

According to a further feature of this aspect of the invention there is provided a method for producing an EphB4 inhibitory effect in a warm-blooded animal, such as man, in need of such treatment which comprises administering to said animal an effective amount of a compound of the formula I, IA, IB, IC or ID, or a pharmaceutically acceptable salt thereof, as defined hereinbefore.

According to a further aspect of the invention there is provided the use of a compound of the formula I, IA, IB, IC or ID, or a pharmaceutically acceptable salt thereof, as defined hereinbefore in the manufacture of a medicament for use in the production of an anti-angiogenic effect in a warm-blooded animal such as man.

According to a further feature of this aspect of the invention there is provided a method for producing an anti-angiogenic effect in a warm-blooded animal, such as man, in need of such treatment which comprises administering to said animal an effective amount of a compound of the formula I, IA, IB, IC or ID, or a pharmaceutically acceptable salt thereof, as defined hereinbefore.

According to an additional feature of this aspect of the invention there is provided a method of treating cancer in a warm-blooded animal, such as man, in need of such treatment which comprises administering to said animal an effective amount of a compound of the formula I, IA, IB, IC or ID, or a pharmaceutically acceptable salt thereof, as defined hereinbefore.

According to a further feature of the invention there is provided a compound of the formula I, IA, IB, IC or ID, or a pharmaceutically acceptable salt thereof, as defined hereinbefore in the manufacture of a medicament for use in the treatment of cancer.

According to an additional feature of this aspect of the invention there is provided a compound of the formula I, IA, IB, IC or ID, or a pharmaceutically acceptable salt thereof, as defined hereinbefore, for use in the treatment of cancer.

According to an additional feature of this aspect of the invention there is provided a compound of the formula I, IA, IB, IC or ID, or a pharmaceutically acceptable salt thereof, as defined hereinbefore, for use in the treatment of solid tumour disease, in particular neuroblastomas, breast, liver, lung and colon cancer or leukemias.

According to an additional feature of this aspect of the invention there is provided the use of a compound of the formula I, IA, IB, IC or ID, or a pharmaceutically acceptable salt thereof, as defined hereinbefore, for use in the manufacture of a medicament for the treatment of cancer.

In a further aspect of the present invention there is provided the use of a compound of the formula I, IA, IB, IC or ID, or a pharmaceutically acceptable salt thereof, as defined hereinbefore, in the manufacture of a medicament for use in the treatment of solid tumour disease, in particular neuroblastomas, breast, liver, lung and colon cancer or leukemias.

In a further aspect of the present invention there is provided a method of treating neuroblastomas, breast, liver, lung and colon cancer or leukemias in a warm-blooded animal, such as man, in need of such treatment which comprises administering to said animal an effective amount of a compound of the formula I, IA, IB, IC or ID, or a pharmaceutically acceptable salt thereof, as defined hereinbefore.

The anti-cancer treatment defined hereinbefore may be applied as a sole therapy or may involve, in addition to the compound of the invention, conventional surgery or radiotherapy or chemotherapy. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the anti-angiogenic treatment defined hereinbefore may be: surgery, radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example antifolates such as fluoropyrimidines like 5-fluorouracil and tegafur, raltitrexed, methotrexate, cytosine arabinoside, hydroxyurea and gemcitabine); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine, taxoids like taxol and taxotere, and polo kinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and bosutinib (SKI-606), and metalloproteinase inhibitors like marimastat and inhibitors of urokinase plasminogen activator receptor function];
(iv) inhibitors of growth factor function: for example such inhibitors include growth factor antibodies and growth factor receptor antibodies [for example the anti-erbB2 antibody trastuzumab and the anti-erbB1 antibodies cetuximab (C225) and panitumumab]; such inhibitors also include, for example, tyrosine kinase inhibitors [for example inhibitors of the epidermal growth factor family (for example EGFR family tyrosine kinase inhibitors such as gefitinib (ZD1839), erlotinib (OSI-774) and CI 1033, and erbB2 tyrosine kinase inhibitors such as lapatinib), inhibitors of the hepatocyte growth factor family, inhibitors of the insulin growth factor receptor, inhibitors of the platelet-derived growth factor family and/or bcr/abl kinase such as imatinib, dasatinib (BMS-354825) and nilotinib (AMN107), inhibitors of cell signalling through MEK, AKT, PI3, c-kit, Flt3, CSF-1R and/or aurora kinases]; such inhibitors also include cyclin dependent kinase inhibitors including CDK2 and CDK4 inhibitors; and such inhibitors also include, for example, inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336);
(v) antiangiogenic agents such as those which inhibit the effects of vascular endothelial growth factor, [for example an anti-vascular endothelial cell growth factor antibody such as bevacizumab (Avastin™) or, for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), or, for example, a compound that works by another mechanism (for example linomide, inhibitors of integrin αvβ3 function and angiostatin)];
(vi) vascular damaging agents such as Combretastatin A4;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination suitable for use in the treatment of cell proliferative disorders (such as solid tumour disease) comprising a compound of formula I, IA, IB, IC or ID as defined hereinbefore and an additional anti-tumour agent as defined hereinbefore.

According to this aspect of the invention there is provided a pharmaceutical product comprising a compound of formula I, IA, IB, IC or ID as defined hereinbefore and an additional anti-tumour agent as defined hereinbefore for the conjoint treatment of cancer.

As stated above the size of the dose required for the therapeutic or prophylactic treatment of a particular cell-proliferation disease will necessarily be varied depending on the host treated, the route of administration and the severity of the illness being treated. A unit dose in the range, for example, 1-100 mg/kg, preferably 1-50 mg/kg is envisaged.

In addition to their use in therapeutic medicine, the compounds of formula I, IA, IB, IC or ID and their pharmaceutically acceptable salts thereof, are also useful as pharmacological tools in the development and standardisation of in vitro and in vivo test systems for the evaluation of the effects of inhibitors of anti-angiogenic activity in laboratory animals such as cats, dogs, rabbits, monkeys, rats and mice, as part of the search for new therapeutic agents.

The invention will now be illustrated in the following Examples in which, generally:

I temperatures are given in degrees Celsius (° C.); operations were carried out at room or ambient temperature, that is, at a temperature in the range of 18 to 25° C.;
(ii) organic solutions were dried over anhydrous magnesium sulfate or anhydrous sodium sulfate; evaporation of solvent was carried out using a rotary evaporator under reduced pressure (600 to 4000 Pascals; 4.5 to 30 mmHg) with a bath temperature of up to 60° C.;
(iii) chromatography means flash chromatography on silica gel; thin layer chromatography (TLC) was carried out on silica gel plates;
(iv) in general, the course of reactions was followed by TLC and/or analytical LC-MS, and reaction times are given for illustration only. The retention times (t_{R}) were measured on a LC/MS Waters 2790/ZMD Micromass system equipped with a Waters Symmetry column (C18, 3.5 μM, 4.6×50 mm); detection UV 254 nM and MS; elution: flow rate 2.5 ml/min, linear gradient from 95% water-5% methanol containing 5% formic acid to 40% water-55% acetonitrile-5% methanol containing 5% formic acid over 3 minutes; then linear gradient to 95% acetonitrile-5% methanol containing 5% formic acid over 1 minute;
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 500 MHz using perdeuterio dimethyl sulfoxide (DMSO-d₆) as solvent unless otherwise indicated; the following abbreviations have been used: s, singlet; d, doublet; t, triplet; q, quartet; m, multiplet; br, broad;
(viii) chemical symbols have their usual meanings; SI units and symbols are used;
(ix) solvent ratios are given in volume:volume (v/v) terms; and
(x) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺ which refers to the protonated mass ion; reference to M⁺ is to the mass ion generated by loss of an electron; and reference to M−H⁺ is to the mass ion generated by loss of a proton;
(xi) unless stated otherwise compounds containing an asymmetrically substituted carbon and/or sulfur atom have not been resolved;
(xii) where a synthesis is described as being analogous to that described in a previous example the amounts used are the millimolar ratio equivalents to those used in the previous example;
(xiii) all microwave reactions were carried out in a Personal Chemistry EMRYS™ Optimizer EXP microwave synthesisor;
(xiv) preparative high performance liquid chromatography (HPLC) was performed on a Waters instrument using the following conditions:

| | |
|---|---|
| Column: | 30 mm × 15 cm Xterra Waters, C18, 5 mm |
| Solvent A: | Water with 1% acetic acid or 2 g/l ammonium |
| | carbonate |
| Solvent B: | Acetonitrile |
| Flow rate: | 40 ml/min |
| Run time: | 15 minutes with a 10 minute gradient from 5-95% B |
| Wavelength: | 254 nm |
| Injection volume | 2.0-4.0 ml; |

In addition, the following abbreviations have been used, where necessary:—

LiHMDS Lithium bis(trimethylsilyl)amide
NaHMDS Sodium bis(trimethylsilyl)amide
DMSO dimethylsulphoxide
NMP 1-methyl-2-pyrrolidinone

### DMA N,N-dimethylacetamide

### DCM Dichloromethane

THF tetrahydrofuran;

### DMF N,N-dimethylformamide;

DTAD di-tert-butyl azodicarboxylate;
DIPEA di-isopropylethylamine;
IPA isopropyl alcohol;
Ether diethyl ether; and
TFA trifluoroacetic acid.

### EXAMPLE 1

### N-(3,5-dimorpholin-4-ylphenyl)-N′-(4-methoxypyridin-2-yl)-N′-methyl-pyrimidine-2,4-diamine

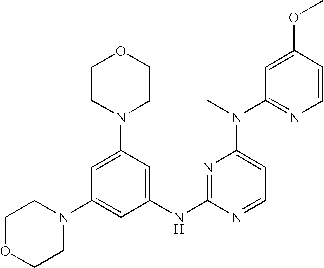

4-Methoxy-N-methyl-pyridin-2-amine (92 mg, 0.75 mmol), 4-chloro-N-(3,5-dimorpholin-4-ylphenyl)pyrimidin-2-amine (188 mg, 0.50 mmol), potassium carbonate (691 mg, 5.01 mmol), bis(dibenzylideneacetone)palladium(0) (14 mg, 0.025 mmol) and 9,9-dimethyl-4,5-bis(diphenylphosphino)xanthene (29 mg, 0.05 mmol) were dissolved in toluene (5 mL). The mixture was degassed, purged with nitrogen and heated at 120° C. for 3 hours. The reaction mixture was filtered off and washed thoroughly with dichloromethane. The filtrate was concentrated to dryness and purified by flash chromatography on silica gel eluting with 1 to 4% methanol in DCM. The solvent was evaporated to dryness to afford the title compound (145 mg, 61%) as a white foam. NMR Spectrum: (CDCl₃) 3.09-3.15 (m 8H), 3.60 (s, 3H), 3.79 (s, 3H), 3.80-3.86 (m, 8H), 6.16 (t, 1H), 6.31 (d, 1H), 6.68 (dd, 1H), 6.80 (d, 2H), 6.84 (d, 1H), 7.24 (bs, 1H), 7.97 (d, 1H), 8.30 (d, 1H); Mass spectrum: MH⁺ 478.

4-Chloro-N-(3,5-dimorpholin-4-ylphenyl)pyrimidin-2-amine used as starting material was made as follows

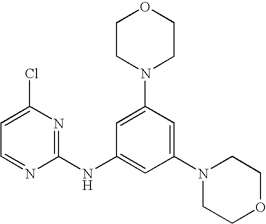

A mixture of 3,5-difluoro-1-nitrobenzene (50 g, 314 mmol) and anhydrous DMSO (25 ml) in morpholine (164 ml, 1.89 mol) was heated at 160° C. for 24 hours. Additional anhydrous DMSO (12.5 ml) was added and the mixture was heated at 160° C. for 66 hours more. After cooling, the mixture was diluted in DCM, washed with water and brine and dried over MgSO4. After evaporation of the solvents, the residue was purified by chromatography on silica gel (eluant: 5% EtOAc in DCM) to give 4-(3-morpholin-4-yl-5-nitro-phenyl)morpholine (53.3 g, 58%) as an orange solid. NMR Spectrum: (DMSOd₆) 3.21 (m, 8H), 3.73 (m, 8H), 6.84 (s, 1H), 7.15 (s, 2H); Mass spectrum: MH⁺ 294.

4-(3-Morpholin-4-yl-5-nitro-phenyl)morpholine (53.3 g, 182 mmol) in ethanol (700 ml) was hydrogenated at atmospheric pressure and room temperature in the presence of 10% palladium on charcoal (5 g) for 17 hours. After filtration of the solids and washing with DMF, the resulting filtrate was concentrated under vacuum. The residue was triturated in ether and dried under vacuum. This solid was solubilised in DCM. The resulting solution was filtered and ether was added. The resulting solid was filtered and dried under high vacuum to give 3,5-dimorpholin-4-ylaniline (46.5 g, 97%) as a beige solid.

NMR Spectrum: (DMSOd₆) 2.97 (m, 8H), 3.68 (m, 8H), 4.74 (s, 2H), 5.69 (s, 2H), 5.73 (s, 1H); Mass spectrum: MH⁺ 264

Sodium hydride (60%, 0.99 g, 24.7 mmol) was added portionwise to a ice-cooled solution of N-(3,5-dimorpholin-4-ylphenyl)formamide (4.5 g, 15 mmol) [prepared by heating 3,5-dimorpholin-4-ylaniline (10 g) in formic acid (100 ml) for 3 h at reflux, evaporation of the solvent, partitioning with ethyl acetate/aq. sodium bicarbonate and chromatography on silica gel (1 to 4% MeOH in DCM)] in THF (130 ml). The mixture was stirred at room temperature for 15 minutes, then cooled at 0° C. 4-Chloro-2-methylsulfonylpyrimidine (3.26 g, 17 mmol, L. Xu et al, J. Org. Chem. 2003, 68, 5388) was added portionwise to the mixture. The reaction was warmed to room temperature and stirred overnight. An aqueous solution of sodium hydroxide (2N, 14 ml) and methanol (20 ml) were added and the mixture stirred for 1 hour. After concentration under vacuum, the residue was dissolved in methylene chloride, washed with water, dried and evaporated to provide a beige solid after trituration in diethyl ether (4.2 g, 73%). NMR Spectrum (500 MHz, DMSO) 3.05-3.07 (m, 8H), 3.72-3.73 (m, 8H), 6.19 (s, 1H), 6.88 (s, 2H), 6.91 (d, 1H), 8.41 (d, 1H), 9.73 (s, 1H); Mass Spectrum MH⁺ 376.

4-Methoxy-N-methyl-pyridin-2-amine used as starting material was made as follows:

Sodium hydride (428 mg, 10.7 mmol, 60% in oil) was added portionwide to an ice-cooled solution of tert-butyl N-(4-methoxypyridin-2-yl)carbamate (2 g, 8.92 mmol) in THF (10 ml).

The mixture was stirred at 0° C. for 15 minutes and methyl iodide (0.691 ml, 10.7 mmol) was added dropwise. The mixture was warmed to room temperature and stirred for 2 hours. After evaporation of the solvents, the mixture was quenched with water and extracted with ether (3×). The organic layers were combined, dried and concentrated. The residue was purified by chromatography on silica gel (eluant: 4% methanol in DCM) to give tert-butyl N-(4-methoxypyridin-2-yl)-N-methyl-carbamate (1.75 g, 82%) as a colorless oil. NMR Spectrum: (CDCl₃) 1.53 (s, 9H), 3.39 (s, 3H), 3.85 (s, 3H), 6.58 (m, 1H), 7.26 (s, 1H), 8.18 (d, 1H).

A solution of tert-butyl N-(4-methoxypyridin-2-yl)-N-methyl-carbamate (1.7 g, 7.13 mmol) in TFA (5 ml) was stirred at room temperature for 3 hours. After evaporation of the solvents, the residue was neutralised by addition of 30% aqueous ammonium hydroxide while cooling the mixture. The resulting mixture was extracted with DCM (3×). The combined organic layers were washed with brine, dried over magnesium sulfate and evaporated to give 4-methoxy-N-methyl-pyridin-2-amine (920 mg, 93%). NMR Spectrum: (CDCl₃) 2.89 (d, 3H), 3.81 (s, 3H), 4.63 (m, 1H), 5.85 (s, 1H), 6.20 (m, 1H), 7.91 (d, 1H); Mass spectrum: MH⁺ 139.

### EXAMPLE 2

Using the procedure described in Example 1, the following compounds were prepared:

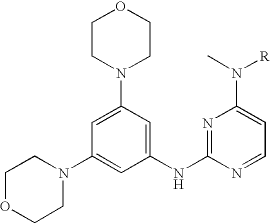

| Ex- | | | | |
|---|---|---|---|---|
| am- | | | Molecular | |
| ple | Name | R | ion (MH⁺) | NMR Spectrum |
| 2a^{a} | N′-(4-chloropyridin-2-yl)-N-(3,5-dimorpholin-4-ylphenyl)-N′-methyl-pyrimidine-2,4-diamine | 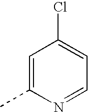 | 482 | (CDCl_{3) 3.10-3.16 (m, 8H),3.62 (s, H), 3.80-3.86 (m, 8H),6.17 (t, 1H), 6.40(d, 1H), 6.78 (d,2H), 7.10 (dd,1H), 7.36 (bs,1H), 7.41 (d, 1H),8.04 (d, 1H), 8.37(d, 1H)} |
| 2b^{b} | N′-(4-chloropyridin-4-yl)-N-(3,5-dimorpholin-4-ylphenyl)-N′-methyl-pyrimidine-2,4-diamine | 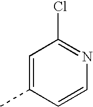 | 482 | (DMSOd6) 2.93-3.01 (m, 8H), 3.48(s, 3H), 3.74-3.71(m, 8H), 6.09 (t,1H), 6.45 (d, 1H),6.85 (d, 2H), 7.41(dd, 1H), 7.48 (d,1H), 8.19 (d, 1H),8.27 (d, 1H), 9.10(s, 1H) |

^{a}The 4-chloro-N-methyl-pyridin-2-amine used as starting material was
made as follows: 4-Chloro-pyridin-2-amine (370 mg, 2.89 mmol) in THF
(5 ml) was added to a solution of LiHMDS (6.36 mmol, 1 M in THF) in
THF (10 ml) cooled at −5° C. The mixture was stirred at −5° C. for 5
minutes and di-tert-butyl dicarbonate (663 mg, 3.04 mmol) in THF (5 ml)
was added. The mixture was stirred at 0° C. for 2 hours. After evapora-
tion of the solvents, water was added. The pH of the solution was adjusted
to 6 by addition of 2N hydrochloric acid and the mixture was extracted
with ethyl acetate (3×). The combined organic layers were washed with
4% aqueous sodium bicarbonate, water and brine, dried over magnesium
sulfate and concentrated. The residue was purified by chromatography on
silica gel (eluant: 10% ethyl acetate in petroleum ether) to give tert-butyl
N-(4-chloropyridin-2-yl)carbamate (465 mg, 70%) as a white solid. NMR
Spectrum: (CDCl₃) 1.54 (s, 9H), 6.96 (m, 1H), 7.91 (bs, 1H), 8.05 (s,
1H), 8.14 (d, 1H). Tert-butyl N-(4-chloropyridin-2-yl)carbamate was
converted into 4-chloro-N-methyl-pyridin-2-amine using the same
procedure as in Example 1, Starting material: tert-butyl N-(4-chloropyri-
din-2-yl)-N-methyl-carbamate (270 mg, 55%), colorless oil: NMR
Spectrum: (CDCl₃) 1.54 (s, 9H), 3.40 (s, 3H), 7.00 (m, 1H), 7.85 (s, 1H),
8.24 (d, 1H). 4-chloro-N-methyl-pyridin-2-amine (15% mg, 96%); Mass
spectrum: MH⁺ 143.
^{b}2-chloro-N-methyl-pyridin-4-amine used as starting material was made
as follows: A mixture of 2,4-dichloropyrimidine (3 g, 20.27 mmol) and
saturated methylamine in methanol (50 ml) was heated in a sealed vessel
at 80° C. for 4 hours. After evaporation of the solvents, the residue was
partitioned between DCM and 30% aqueous ammonium hydroxide and
extracted with DCM (3×). The combined organic layers were dried over
magnesium sulfate to give a yellow oil which was left for 2 days. Partial
crystallisation of the oil occurred. The resulting solid was filtered and
washed with pentane to give 2-chloro-N-methyl-pyridin-4-amine (1.1 g,
38%) as orange crystals. NMR Spectrum: (CDCl₃) 2.86 (d, 3H), 4.47 (bs,
1H), 6.36 (m, 1H), 6.44 (s, 1H), 7.93 (d, 1H). Mass spectrum: MH⁺ 143.

### EXAMPLE 3

### N′-(5-chloropyridin-3-yl)-N-(3,5-dimorpholin-4-ylphenyl)-N′-methyl-pyrimidine-2,4-diamine

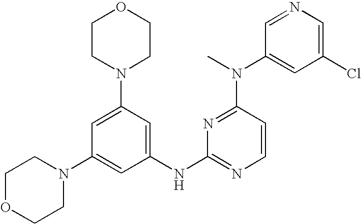

A mixture of 4-chloro-N-(3,5-dimorpholin-4-ylphenyl)-N-[(4-methoxyphenyl)methyl]pyrimidin-2-amine (150 mg, 0.30 mmol), 3-amino-5-chloropyridine (42 mg, 0.33 mmol) and 4M hydrogen chloride in dioxane (0.1 ml) in 2-pentanol (2 ml) in a sealed tube was heated at 120° C. for 3 hours in a Personal Chemistry EMRYS™ Optimizer EXP microwave synthesisor. After cooling, the solvent was evaporated to dryness. The residue was dissolved in DCM (3 ml). 7N Methanolic ammonia (0.3 ml) was added. The insoluble was removed by filtration and the resulting solution was evaporated to dryness.

The residue was dissolved in DMF (2 ml). Sodium hydride (17 mg, 0.42 mmol, 60% in oil) was added and the mixture was stirred for 1 hour at room temperature followed by addition of methyl iodide (19 μl, 0.3 mmol). The resulting mixture was stirred at room temperature for 24 hours and was evaporated to dryness.

The residue (111 mg) was dissolved in trifluoroacetic acid (2 ml) and anisole (1 drop). The mixture was stirred at 130° C. for 30 minutes in a Personal Chemistry EMRYS™ Optimizer EXP microwave synthesisor. The mixture was evaporated to dryness. 7N methanolic ammonia (1 ml) was slowly added. The insoluble was removed by filtration and the resulting solution was evaporated to dryness. The residue was purified by chromatography on silica gel (eluant: 0% to 6% methanol in DCM) to give the title compound (48 mg, 54%) as a solid.

NMR Spectrum: (DMSOd₆) 2.95-3.02 (m, 8H), 3.45 (s, 3H), 3.66-3.74 (m, 8H), 6.03 (d, 1H), 6.09 (t, 1H), 6.87 (d, 2H), 8.01 (d, 1H), 8.05 (dd, 1H), 8.52 (d, 1H), 8.60 (d, 1H), 8.92 (s, 1H); Mass spectrum: MH⁺ 482.

The 4-chloro-N-(3,5-dimorpholin-4-ylphenyl)-N-[(4-methoxyphenyl)methyl]pyrimidin-2-amine used as starting material was made as follows:

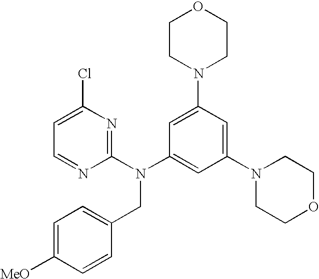

Sodium hydride (1.66 g, 41.6 mmol, 60% in oil) was added portionwise to an ice-cooled solution of 4-chloro-N-(3,5-dimorpholin-4-ylphenyl)pyrimidin-2-amine (13 g, 34.6 mmol, Example 1, Starting material) in THF (100 ml). After stirring 2 hours at room temperature, 4-methoxybenzyl bromide (6.57 ml, 45.1 mmol) and potassium iodide (100 mg) were added to the mixture followed by DMF (10 ml). The resulting mixture was stirred at room temperature for 15 hours. The mixture was partitioned with saturated aqueous ammonium chloride and ethyl acetate, and further extracted with ethyl acetate. The organic layers were combined and dried over magnesium sulfate. After evaporation of the solvents, the residue was purified by chromatography on silica gel (eluant: 40% to 100% ethyl acetate in petroleum ether) to give the title compound (12.37 g, 72%) as a white solid after trituration in ether/petroleum ether. NMR Spectrum: (DMSOd₆) 3.01 (m, 8H), 3.70 (m, 11H), 5.05 (s, 2H), 6.23 (s, 2H), 6.33 (s, 1H), 6.82 (m, 3H), 7.17 (d, 2H), 8.29 (d, 1H); Mass spectrum: MH⁺ 496.

### EXAMPLE 4

### N′-(6-chloropyridin-2-yl)-N-(3,5-dimorpholin-4-ylphenyl)-N′-methyl-pyrimidine-2,4-diamine

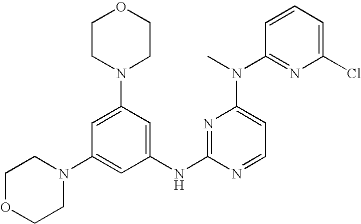

A mixture of 2-chloro-N-(6-chloropyridin-2-yl)-N-methyl-pyrimidin-4-amine (87 mg, 0.34 mmol), 3,5-dimorpholin-4-ylaniline (95 mg, 0.34 mmol), 6N HCl in 2-propanol (2 drops) and 2-propanol (5 mL) is heated at reflux for 2 hours. The reaction mixture was cooled to room temperature and diluted with 30% aqueous ammonium hydroxide. The mixture was extracted with methylene chloride (2×). After evaporation, the crude material was purified by chromatography on silica gel (0 to 3% methanol in DCM) to give the title compound (110 mg, 67%) as a pink solid. NMR Spectrum (DMSO-d6) 2.96-3.02 (m, 8H), 3.52 (s, 3H), 3.67-3.72 (m, 8H), 6.10 (t, 1H), 6.51 (d, 1H), 6.88 (d, 2H), 7.25 (d, 1H), 7.49 (d, 1H), 7.80 (dd, 1H), 8.15 (d, 1H), 9.05 (s, 1H); Mass Spectrum: MH⁺ 482.

2-Chloro-N-(6-chloropyridin-2-yl)-N-methyl-pyrimidin-4-amine used as starting material was made as follows:

NaHMDS (1.5 ml, 1.5 mmol, 1N in THF) was added dropwise to a mixture of 2-chloro-6-methylaminopyridine (142 mg, 1 mmol, German Patent, DE3318560, p 9) and 2,4-dichloropyrimidine (222 mg, 1.5 mmol) in THF (20 ml) cooled at −20° C. The mixture was stirred at −20° C. for 2 hours. Acetic acid (a few drops) were added and the mixture was concentrated. The mixture was taken in DCM, filtered and concentrated. The residue was purified by chromatography on silica gel (eluant: 40% to 50% ethyl acetate in petroleum ether) to give 2-chloro-N-(6-chloropyridin-2-yl)-N-methyl-pyrimidin-4-amine (86 mg, 34%). NMR Spectrum: (CDCl₃) 3.61 (s, 3H), 6.93 (d, 1H), 7.18 (d, 1H), 7.28 (m, 1H), 7.72 (t, 1H), 8.17 (d, 1H); Mass spectrum: MH⁺ 255.

### EXAMPLE 5

### N-(3,5-dimorpholin-4-ylphenyl)-N′-(6-methoxypyridin-2-yl)-N′-methyl-pyrimidine-2,4-diamine

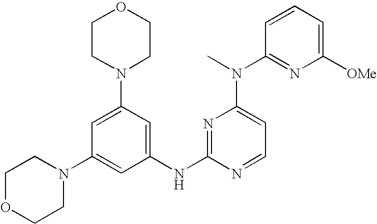

According to procedure of Example 4, 2-chloro-N-(6-methoxypyridin-2-yl)-N-methyl-pyrimidin-4-amine (70 mg, 0.28 mmol) and 3,5-dimorpholin-4-ylaniline (76 mg, 0.29 mmol) were reacted to give the title compound (85 mg, 63%) as a pink solid. NMR Spectrum: (DMSOd₆) 2.99-3.06 (m, 8H), 3.54 (s, 3H), 3.68-3.73 (m, 8H), 3.84 (s, 3H), 6.11 (t, 1H), 6.46 (d, 1H), 6.61 (d, 1H), 6.94 (d, 2H), 7.00 (d, 1H), 7.72 (dd, 1H), 8.04 (d, 1H), 8.96 (s, 1H); Mass spectrum: MH⁺ 478.

2-chloro-N-(6-methoxypyridin-2-yl)-N-methyl-pyrimidin-4-amine was made from 2-chloro-6-methylaminopyridine (German Patent, DE3318560, p 8) according to procedure of Example 4, starting material: 72 mg, 26%; NMR Spectrum: (DMSOd₆) 3.48 (s, 3H), 3.82 (s, 3H), 6.74 (d, 1H), 7.02 (m, 2H), 7.82 (t, 1H), 8.16 (d, 1H); Mass spectrum: MH⁺ 251.

### EXAMPLE 6

### N4-(6-chloropyridin-3-yl)-N2-(3,5-dimorpholinophenyl)-N4-methylpyrimidine-2,4-diamine

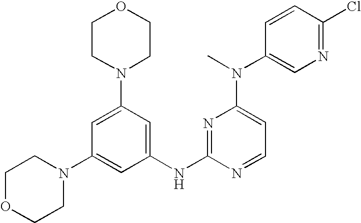

A suspension of 4-chloro-N-(3,5-dimorpholinophenyl)pyrimidin-2-amine (150 mg, 0.40 mmol), 6-chloro-N-methylpyridin-3-amine (62.6 mg, 0.44 mmol; Zakrzewski P. et al., Synthesis, 1999, 11, 1893) and HCl/dioxane 4N (210 μl, 0.84 mmol) in 2-pentanol (1.5 ml) was stirred at 120° C. over a period of 2 hours under nitrogen. The reaction mixture was allowed to cool to room temperature. The resulting precipitate was collected by filtration, washed with 2-pentanol and ether, and diluted with DCM (5 ml) and 5N methanolic ammonia (1 ml). The mixture was concentrated to dryness, diluted with DCM (10 ml) and filtered. The filtrate was concentrated and purified by flash chromatography on silica gel eluting with 2% methanol in dichloromethane. After evaporation, the residue was tritured with ether and dried at 50° C. overnight to afford the title compound (135 mg, 70.2%) as a white solid. NMR Spectrum: (DMSOd₆) 2.94-3.03 (m, 8H), 3.53 (s, 3H), 3.67-3.74 (m, 8H), 6.01 (d, 1H), 6.09 (t, 1H), 6.87 (d, 2H), 7.58 (d, 1H), 7.90 (dd, 1H), 8.00 (d, 1H), 8.46 (d, 1H), 8.89 (s, 1H); Mass spectrum: MH⁺ 482.

### EXAMPLE 7

Using the procedure described in Example 3,4-chloro-N-(3,5-dimorpholin-4-ylphenyl)-N-[(4-methoxyphenyl)methyl]pyrimidin-2-amine was reacted with the corresponding aminopyridine to give the following compounds:

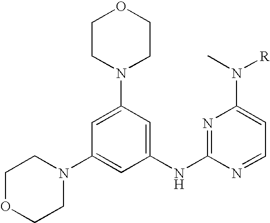

| Ex- | | | | |
|---|---|---|---|---|
| am- | | | Molecular | NMR Spectrum |
| ple | Name | R | ion (MH⁺) | (DMSOd6) |
| 7a | N-(3,5-dimorpholin-4-ylphenyl)-N′-methyl-N′-(6-methylpyridin-yl)pyrimidine-2,4-diamine | 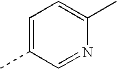 | 462 | 2.52 (s partiallyhidden by DMSO,3H), 2.97-3.05(m, 8H), 3.42 (s,3H), 3.68-3.73(m, 8H), 5.78 (d,1H), 6.10 (t, 1H),6.93 (d, 2H), 7.35(d, 1H), 7.69 (dd,1H), 7.90 (d, 1H),8.44 (d, 1H), 8.86(s, 1H) |
| 7b^{a} | N-(3,5-dimorpholin-4-ylphenyl)-N′-(5-methoxypyridin-3-yl)-N′-methyl-pyrimidine-2,4-diamine | 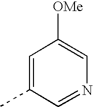 | 478 | 2.98-3.04 (m,8H), 3.45 (s, 3H),3.68-3.73 (m,8H), 3.82 (s, 3H),5.88 (d, 1H), 6.10(t, 1H), 6.93 (d,2H), 7.46 (dd,1H), 7.94 (d, 1H),8.19 (d, 1H), 8.23(d, 1H), 8.89 (s,1H) |
| 7c^{b} | N′-(2,5-dimethylpyridin-3-yl)-N-(3,5-dimorpholin-4-ylphenyl)-N′-methyl-pyrimidine-2,4-diamine | 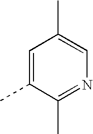 | 476 | (DMSOd6 at323° K.) 2.24 (s,3H), 2.30 (s, 3H),3.00-3.10 (m, 8H), 3.34 (s, 3H),3.67-3.76 (m,8H), 5.41 (bs,1H), 6.09 (t, 1H),6.95 (s, 2H), 7.51s, 1H), 7.85 (d,1H), 8.32 (s, 1H),8.65 (bs, 1H) |
| 7d^{c} | N-(3,5-dimorpholin-4-ylphenyl)-N′-(5-methoxy-2-methyl-pyridin-3-yl)-N′-methyl-pyrimidine-2,4-diamine | 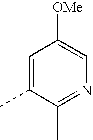 | 492 | (DMSOd6 at323° K.) 2.21 (s,3H), 2.99-3.11(m, 8H), 3.37 (s,3H), 3.67-3.78(m, 8H), 3.82 (s,3H), 5.46 (bs,1H), 6.09 (s, 1H),6.95 (s, 2H), 7.35(d, 1H), 7.86 (d,1H), 8.22 (d, 1H),8.66 (s, 1H) |

^{a}4-Chloro-N-(3,5 -dimorpholinophenyl)-N-(4-methoxybenzyl)pyrimidin-2-
amine (200 mg, 0.40 mmol) and 5-methoxypyridin-3-amine (55.1 mg,
0.44 mmol; Tamura Y. et al., J. Org Chem., 1981, 46, 3564) were reacted
in the first step using Buchwald conditions: potassium carbonate (557 mg,
4.03 mmol), bis(dibenzylideneacetone)palladium(0) (23.2 mg, 0.04 mmol)
and 9,9-dimethyl-4,5-bis(diphenylphosphino)xanthene (23.3 mg, 0.04
mmol) in toluene (3 ml) at reflux under argon and heated to reflux for 5 h.
Additional 5-methoxypyridin-3-amine (10 mg, 0.08 mmol), 9,9-dimethyl-
4,5-bis(diphenylphosphino)xanthene (11.7 mg, 0.02 mmol) and bis(di-
benzylideneacetone)palladium(0) (11.6 mg, 0.02 mmol) were added to the
reaction mixture and the reaction mixture was heated at 120° C. for 18
hours. The reaction mixture was cooled, diluted with DCM (5 ml) and
filtered. The filtrate was concentrated to dryness and purified by flash
chromatography on silica gel eluting with 2 to 4% methanol in dichloro-
methane to give N2-(3,5-dimorpholinophenyl)-N2-(4-methoxybenzyl)-N4-
(5-methoxypyridin-3-yl)pyrimidine-2,4-diamine (120 mg, 51%); Mass
spectrum: MH⁺ 584 which was alkylated with methyl iodide and de-
protected using TFA according to the procedure described in Example 3.
^{b}4-Chloro-N-(3,5-dimorpholinophenyl)-N-(4-methoxybenzyl)pyrimidin-2-
amine (200 mg, 0.40 mmol) and 2,5-dimethylpyridin-3-amine (81 mg,
0.67 mmol) were reacted in the first step using Buchwald conditions:
9,9-dimethyl-4,5-bis(diphenylphosphino)xanthene (35 mg, 0.06 mmol),
tris(dibenzylideneacetone)dipalladium (13.8 mg, 0.02 mmol) and 1,8-
diazabicyclo [5.4.0] undec-7-ene (226 μl, 1.51 mmol) in DME (3 ml)
heated under argon at 130° C. over a period of 35 minutes in a Personal
Chemistry EMRYS ™ Optimizer EXP microwave synthesisor.
^{c}4-Chloro-N-(3,5-dimorpholinophenyl)-N-(4-methoxybenzyl)pyrimidin-2-
amine (200 mg, 0.40 mmol) and 2-methyl-5-methoxypyridin-3-amine were
reacted in the same conditions as in Note b above. 2-Methyl-5-methoxy-
pyridin-3 amine was made as follows: 2-Chloro-5-methoxypyridin-3-
amine (250 mg, 1.58 mmol; Barlaam B. et al., Bioorg. Med. Chem. Lett.,
2005, 15, 5446), trimethylboroxine (244 μl, 1.75 mmol), {1,1′-bis(di-
phenylphosphene)ferrocene}palladium chloride complex with dichloro-
methane (63.7 mg, 0.08 mmol) and potassium carbonate (654 mg, 4.73
mmol) were suspended in DME (5 ml) and sealed into a microwave tube.
The reaction was degased, purged with argon and heated to 120° C. over
a period of 30 minutes in a Personal Chemistry EMRYS ™ Optimizer
EXP microwave synthesisor. The reaction mixture was diluted with
dichloromethane. The salts were filred and the filtrate was purified by
flash chromatography on silica gel eluting with 0 to 3% methanol in
dichloromethane to afford 5-methoxy-2-methylpyridin-3-amine (191 mg,
122%) as a green solid. NMR Spectrum: (CDCl3) 2.34 (s, 3H), 3.60 (bs,
2H), 3.80 (s, 3H), 6.51 (d, 1H), 7.67 (d, 1H); Mass spectrum: MH⁺ 139

### EXAMPLE 8

Using the procedure described in Example 6, 4-chloro-N-(3,5-dimorpholinophenyl)pyrimidin-2-amine was reacted with the corresponding aminopyridine to give the following compounds:

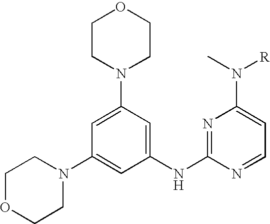

| | | | Molec- | |
|---|---|---|---|---|
| Ex- | | | ular | |
| am- | | | ion | NMR Spectrum |
| ple | Name | R | (MH⁺) | (DMSOd6) |
| 8a^{a} | N′-(6-chloro-5-methoxy-pyridin-3-yl)-N-(3,5-dimorpholin-4-ylphenyl)-N′-methyl-pyrimidine-2,4-diamine | 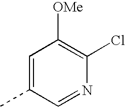 | 512 | (DMSOd6 at 297° K.) 2.95-3.02 (m,8H), 3.46 (s, 3H),3.65-3.74 (m,8H), 3.84 (s, 3H),6.03 (d, 1H), 6.10(t, 1H), 6.88 (d,2H), 7.66 (d, 1H),7.99 (d, 1H), 8.02(d, 1H), 8.91 (s,1H) |
| 8b^{b} | N′-(6-chloro-5-methyl-pyridin-3-yl)-N-(3,5-dimorpholin-4-ylphenyl)-N′-methyl-pyrimidine-2,4-diamine | 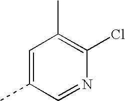 | 496 | (DMSOd6 at297° K.) 2.33(s, 3H), 2.95-3.04(m, 8H), 3.43 (s,3H), 3.66-3.75(m, 8H), 5.97 (d,1H), 6.09 (t, 1H),6.89 (d, 2H), 7.88(d, 1H), 7.98 (d,1H), 8.30 (d, 1H),8.89 (s, 1H) |

^{a}2-Chloro-3-methoxypyridin-5-amine (500 mg, 3.15 mmol; Eastman
Kodak U.S. Pat. No. US4204870, column 38) and triethyl orthoformate
(3.04 ml, 18.29 mmol) under a dry atmosphere was stirred at 145° C. for 1
hour. The solution was cooled to room temperature and concentrated. The
residue was dried under high vacuum overnight, dissolved in ethanol (5
ml). Sodium borohydride (143 mg, 3.78 mmol) was added to this
solution. The resulting mixture was stirred at 80° C. for 1 hour. The
reaction mixture was concentrated and quenched with ice and water (20
ml). Concentrated hydrochloric acid (0.5 ml) was added. The pH of
solution was adjusted to pH 7 with sodium bicarbonate and the mixture
was extracted with ethyl acetate (×3). The combined organic phases were
washed with water, dried over sodium sulfate and concentrated. The
crude product was purified by flash chromatography on silica gel eluting
with 25 to 30% ethyl acetate in petroleum ether. The solvent was evapo-
rated to dryness to afford 6-chloro-5-methoxy-N-methylpyridin-3-amine
(357 mg, 65.6%) as a beige solid. NMR Spectrum: (DMSOd6) 2.71 (d,
3H), 3.82 (s, 3H), 6.07 (q, 1H), 6.65 (d, 1H), 7.28 (d, 1H); Mass
spectrum: MH⁺ 173.
^{b}Using the same procedure as above, 6-chloro-5-methylpyridin-3-
amine (500 mg, 3.51 mmol) gave 6-chloro-N,5-dimethylpyridin-3-amine
(422 mg, 77%) as a beige solid. NMR Spectrum: (DMSOd6) 2.22 (s, 3H),
2.68 (d, 3H), 5.97 (q, 1H), 6.89 (d, 1H), 7.53 (d, 1H); Mass spectrum:
MH⁺ 157.

### EXAMPLE 9

N′-(2-chloro-5-methoxy-pyridin-3-yl)-N-(3,5-dimorpholin-4-ylphenyl)-N′-methyl-pyrimidine-2,4-diamine

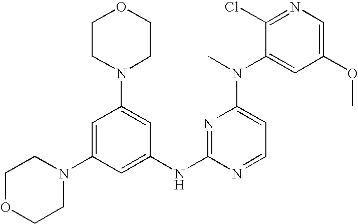

A mixture of 2-chloro-N-(2-chloro-5-methoxypyridin-3-yl)-N-methylpyrimidin-4-amine (230 mg, 0.81 mmol), 3,5-dimorpholinoaniline (212 mg, 0.81 mmol) and 4M hydrochloric acid in dioxane (0.141 ml, 0.56 mmol) in 2-propanol (10 ml) was a stirred over a period of 4 hours at reflux. The reaction mixture was concentrated to dryness, basified with a 15% aqueous solution of ammonia and extracted with dichloromethane. The organic phase was washed with water and brine, dried over magnesium sulfate, concentrated and purified by flash chromatography on silica gel eluting with 1 to 4% methanol in dichloromethane. The solvent was evaporated to dryness. The residue was triturated with diethyl ether/pentane to give a solid which was collected by filtration and dried under vacuum to give the title compound (161 mg, 39%) as a beige solid. NMR Spectrum: (DMSO-d6 at 323° K) 2.99-3.07 (m, 8H), 3.35 (s, 3H), 3.67-3.76 (m, 8H), 3.86 (s, 3H), 5.64 (bs, 1H), 6.09 (s, 1H), 6.93 (s, 2H), 7.66 (d, 1H), 7.92 (d, 1H), 8.18 (d, 1H), 8.69 (s, 1H); Mass spectrum: MH⁺ 512.

2-Chloro-N-(2-chloro-5-methoxypyridin-3-yl)-N-methylpyrimidin-4-amine used as starting material was prepared as follows.

According to the procedure described in Example 8, Note a, 2-chloro-5-methoxypyridin-3-amine (300 mg, 1.89 mmol; Barlaam B. et al., Bioorg. Med. Chem. Lett., 2005, 15, 5446)) was coverted into 2-chloro-5-methoxy-N-methylpyridin-3-amine (273 mg, 84%) as a colorless oil. NMR Spectrum: (CDCl3) 2.88 (d, 3H), 3.85 (s, 3H), 4.38 (bs, 1H), 6.43 (d, 1H), 7.41 (d, 1H); Mass spectrum: MH⁺ 173.

Sodium bis(trimethylsilyl)amide (3.05 ml, 3.05 mmol, 1M in THF), was added dropwise to a stirred solution of 2-chloro-5-methoxy-N-methylpyridin-3-amine (247 mg, 1.4 mmol) dissolved in THF (10 ml) at 0° C. under argon. The resulting solution was stirred at 0° C. for 15 minutes then room temperature for 10 minutes. The reaction mixture was cooled to 0° C., 2,4-dichloropyrimidine (227 mg, 1.52 mmol) was added in one portion. The reaction mixture was stirred at 0° C. for 30 minutes, then at room temperature for 30 minutes. The mixture was quenched with a saturated aqueous solution of ammonium chloride and extracted with ethyl acetate. The combined organic phases were washed with a 4% aqueous solution of citric acid, a saturated aqueous solution of sodium hydrogencarbonate, water, brine, dried over magnesium sulfate and concentrated to dryness. The crude product was purified by flash chromatography on silica gel eluting with 20 to 60% ethyl acetate in petroleum ether. The solvent was evaporated to dryness to afford 2-chloro-N-(2-chloro-5-methoxypyridin-3-yl)-N-methylpyrimidin-4-amine (233 mg, 53.6%) as a white solid. NMR Spectrum: (DMSO-d6 at 323° K) 3.34 (s, 3H), 3.87 (s, 3H), 6.29 (bs, 1H), 7.72 (s, 1H), 8.11 (bs, 1H), 8.21 (d, 1H); Mass spectrum: MH⁺ 285.

### EXAMPLE 10

### (6-((2-(3,5-dimorpholinophenylamino)pyrimidin-4-yl)(methyl)amino)-5-methylpyridin-2-yl)methanol

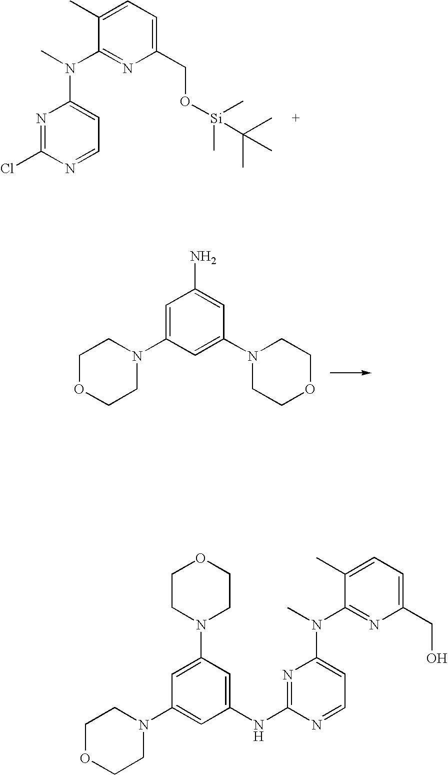

N-(6-((tert-butyldimethylsilyloxy)methyl)-3-methylpyridin-2-yl)-2-chloro-N-methylpyrimidin-4-amine (150 mg, 0.40 mmol), 3,5-dimorpholin-4-ylaniline (105 mg, 0.40 mmol) and hydrochloric acid 4M in dioxane (140 μl, 0.56 mmol) were dissolved in 2-pentanol (4 ml) and sealed into a microwave tube. The reaction was heated at 130° C. over a period of 35 minutes in a microwave reactor. An aqueous solution of HCl (2N; 700 μl) was added and the sealed tube was heated at 80° C. for 5 minutes. The reaction mixture was concentrated to dryness, diluted with dichloromethane, a few drops of a solution 7N of NH₃ in methanol were added, the salts were filtered off and the filtrate was concentrated to dryness. The crude product was purified by flash chromatography on silica gel eluting with 1 to 5% methanol in dichloromethane. The solvent was evaporated to dryness, the resulting gum was dissolved in the minimum volume of dichloromethane and this solution was added dropwise to pentane to give a solid which was collected by filtration and dried under vacuum to give (6-((2-(3,5-dimorpholinophenylamino)pyrimidin-4-yl)(methyl)amino)-5-methylpyridin-2-yl)methanol (99 mg, 50.3%) as a pale beige solid. Mass Spectrum: M+H⁺ 492. NMR Spectrum (DMSO-d6 at 323° K): 2.10 (s, 3H), 3.01-3.08 (m, 8H), 3.37 (s, 3H), 3.67-3.75 (m, 8H), 4.51 (d, 2H), 5.27 (t, 1H), 5.46 (d, 1H), 6.09 (t, 1H), 6.94 (d, 2H), 7.41 (d, 1H), 7.79 (d, 1H), 7.86 (d, 1H), 8.65 (s, 1H)

The N-(6-((tert-butyldimethylsilyloxy)methyl)-3-methylpyridin-2-yl)-2-chloro-N-methylpyrimidin-4-amine used as starting material was made as follows:—

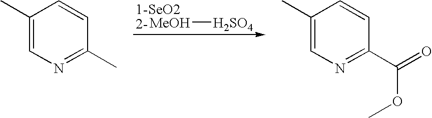

A mixture of 2,5-lutidine (11 g, 102.66 mmol) and selenium (IV) oxide (17.09 g, 153.99 mmol) in pyridine (50 ml) was stirred at 115° C. for 20 hours. The reaction mixture was allowed to warm to room temperature and filtered. The solid material was washed with pyridine (2×10 ml) and water (2×10 ml) and the filtrate was evaporated in vacuo. MeOH (250 ml) and concentrated sulfuric acid (17.11 ml, 615.94 mmol) were added and the resulting mixture was heated at reflux for 8 hours. The mixture was cooled to room temperature and a saturated aqueous solution of sodium hydrogencarbonate was added until pH became basic. The methanol was evaporated in vacuo, water was added and extracted with diethyl ether (150×3 ml). The combined organic phases were washed with brine, dried over magnesium sulfate and concentrated to afford the desired methyl 5-methylpicolinate (6.30 g, 40.6%) as a orange crystalline solid. The crude product was used without further purification for the next step.

Mass Spectrum: M+H⁺ 152. NMR Spectrum (CDCl₃): 2.42 (s, 3H), 4.00 (s, 3H), 7.64 (dd, 1H), 8.04 (d, 1H), 8.57 (d, 1H)

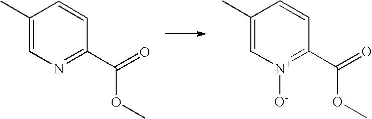

3-chloroperoxybenzoic acid (15.41 g, 62.52 mmol) was added portionwise to a stirred solution of methyl 5-methylpicolinate (6.3 g, 41.68 mmol) dissolved in CH₂Cl₂ (200 ml) over a period of 5 minutes at 20° C. The resulting mixture was stirred at 20° C. overnight. The reaction mixture was quenched with a saturated aqueous Na₂SO₃ (75 ml). The organic phase was collected and washed with a saturated aqueous solution of sodium hydrogencarbonate (100 ml), dried over magnesium sulfate and concentrated to afford the desired 2-(methoxycarbonyl)-5-methylpyridine 1-oxide (5.00 g, 71.8%) as an orange oil. The crude product was used without further purification for the next step. Mass Spectrum: M+H⁺ 168. NMR Spectrum (CDCl₃): 2.34 (s, 3H), 3.99 (s, 3H), 7.10 (d, 1H), 7.56 (d, 1H), 8.13 (s, 1H)

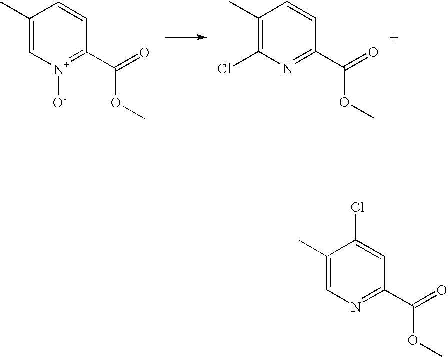

Phosphorus oxychloride (21 ml, 229.40 mmol) was added dropwise to a stirred solution of 2-(methoxycarbonyl)-5-methylpyridine 1-oxide (4.2 g, 25.13 mmol) dissolved in chloroform (16 ml) over a period of 5 minutes. The resulting solution was stirred at 80° C. overnight. The mixture was cooled to room temperature and was added dropwise to a ice cold 10% aqueous solution of K₃CO₃. K₂CO₃ solid was then added to adjust the pH to 7 and the aqueous phase extracted with dichloromethane (3×10 ml). The combined organic phases were washed with brine, dried over magnesium sulfate and concentrated to afford the crude product which was purified by flash chromatography on silica gel eluting with 15 to 30% ethyl acetate in petroleum ether. The solvent was evaporated to dryness to afford methyl 6-chloro-5-methylpicolinate (2.72 g, 58.3%) as a white solid and methyl 4-chloro-5-methylpicolinate (1.000 g, 21.44%) as a white solid. NMR Spectrum (CDCl3): 2.47 (s, 3H), 3.99 (s, 3H), 7.70 (d, 1H), 7.99 (d, 1H)

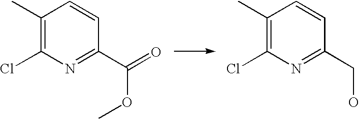

Sodium borohydride (1.005 g, 26.56 mmol) was added to a stirred solution of methyl 6-chloro-5-methylpicolinate (2.9 g, 15.62 mmol) dissolved in ethanol (35 ml) at 20° C. and the resulting suspension stirred overnight. The reaction mixture was concentrated to dryness, poured into brine and extracted with ethyl acetate (3×50 ml). The combined organic phases were washed with brine, dried over magnesium sulfate and concentrated to afford the crude product (6-chloro-5-methylpyridin-2-yl)methanol (2.400 g, 97%) as a clear colorless oil. Mass Spectrum: M+H⁺ 158. NMR Spectrum (CDCl3): 2.38 (s, 3H), 2.95 (bs, 1H), 4.71 (s, 2H), 7.15 (d, 1H), 7.55 (d, 1H)

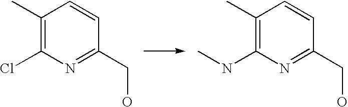

A mixture of (6-chloro-5-methylpyridin-2-yl)methanol (1.1 g, 6.98 mmol) and methylamine (40% solution in water, 2.417 ml, 27.92 mmol) was stirred in sealed tube at 100° C. for 20 hrs. The reaction mixture was concentrated, diluted with ethyl acetate, washed with brine, dried over sodium sulfate and concentrated to afford the crude product. The crude product was purified by flash chromatography on silica gel (15-40 μm) eluting with 30 to 60% ethyl acetate in petroleum ether. The solvent was evaporated to dryness to afford (5-methyl-6-(methylamino)pyridin-2-yl)methanol (0.507 g, 47.7%) as a white solid. Mass Spectrum: M+H⁺ 153. NMR Spectrum (DMSO-d6): 2.00 (s, 3H), 2.80 (d, 3H), 4.35 (d, 2H), 5.03 (t, 1H), 5.82 (q, 1H), 6.51 (d, 1H), 7.17 (d, 1H).

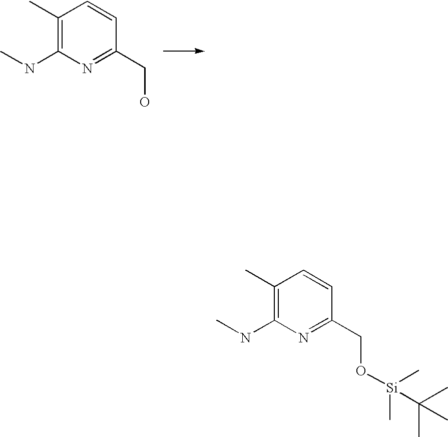

Sodium hydride (60% in oil, 57.8 mg, 1.45 mmol) was added in one portion to a stirred solution of (5-methyl-6-(methylamino)pyridin-2-yl)methanol (200 mg, 1.31 mmol) dissolved in THF (8 ml) at 0° C. under argon. The resulting mixture was stirred at 20° C. for 15 minutes. The mixture was cooled in an ice bath and tert-butyldimethylsilyl chloride (248 μl, 1.45 mmol) was added in one portion. The mixture was stirred at 20° C. overnight. The reaction mixture was concentrated to dryness, diluted with water and extracted with ethyl acetate. The organic phase was washed with brine, dried over magnesium sulfate and concentrated to afford the crude product which was purified by flash chromatography on silica gel (15-40 μm) eluting with 6% ethyl acetate in petroleum ether. The solvent was evaporated to dryness to afford 6-((tert-butyldimethylsilyloxy)methyl)-N,3-dimethylpyridin-2-amine (253 mg, 72.3%) as a white solid. NMR Spectrum (DMSO-d₆): 0.10 (s, 6H), 0.92 (s, 9H), 2.02 (s, 3H), 2.92 (d, 3H), 4.55 (s, 2H), 5.88 (q, 1H), 6.50 (d, 1H), 7.20 (d, 1H).

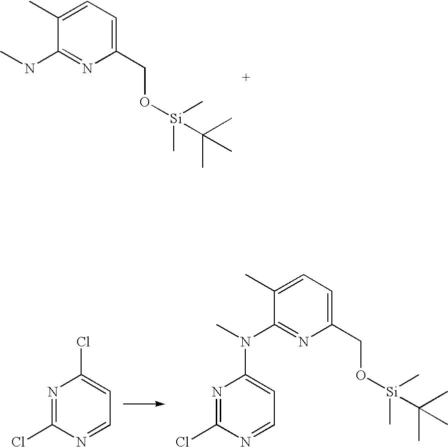

A 1M solution of sodium bis(trimethylsilyl)amide in THF (0.976 ml, 0.98 mmol), was added dropwise to a stirred solution of 6-((tert-butyldimethylsilyloxy)methyl)-N,3-dimethylpyridin-2-amine (200 mg, 0.75 mmol) and 2,4-dichloropyrimidine (224 mg, 1.50 mmol) dissolved in THF (10 ml) at −20° C. under argon. The resulting solution was stirred at −20° C. for 12 hours and at room temperature for 2 hours. The reaction mixture was quenched with a few drops of acetic acid, concentrated to dryness and purified by flash chromatography on silica gel (15-40-μm) eluting with 5 to 25% ethyl acetate in petroleum ether. The solvent was evaporated to dryness to afford N-(6-((tert-butyldimethylsilyloxy) methyl)-3-methylpyridin-2-yl)-2-chloro-N-methylpyrimidin-4-amine (155 mg, 54.5%) as a colorless gum. Mass Spectrum: M+H⁺ 379. NMR Spectrum (DMSO-d₆ at 323° K): 0.12 (s, 6H), 0.94 (s, 9H), 2.13 (s, 3H), 3.35 (s, 3H), 4.74 (s, 2H), 6.20 (bs, 1H), 7.45 (d, 1H), 7.89 (d, 1H), 8.08 (d, 1H).

### EXAMPLE 11

### N-[3,5-di(morpholin-4-yl)phenyl]-N′-methyl-N′-(6-methylpyridin-2-yl)pyrimidine-2,4-diamine

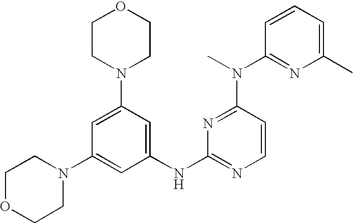

4-Chloro-N-(3,5-dimorpholin-4-ylphenyl)pyrimidin-2-amine (described in Example 1, 200 mg, 0.53 mmol), 6-methyl-2-methylaminopyridine (98 mg, 0.8 mmol), potassium carbonate (736 mg, 5.3 mmol), Pd2 dba3 (16 mg, 0.027 mmol) and Xantphos (31 mg, 0.053 mmol) were mixed in toluene (5 ml). The mixture was degassed with nitrogen and heated in a sealed tube at 120° C. for 3 hours. After filtration, the toluene was evaporated and the residue purified on a preparative HPLC-MS system (Column: C18, 5 microns, 19 mm diameter, 100 mm length, elution with a gradient of water and acetonitrile containing 2 g/l of ammonium carbonate) to give 55 mg of the title compound (22% yield). NMR Spectrum (500 MHz, DMSO-d6) 2.45 (s, 3H), 3.01 (m, 8H), 3.51 (s, 3H), 3.70 (m, 8H), 6.10 (s, 1H), 6.30 (d, 1H), 6.93 (d, 2H), 7.07 (d, 1H), 7.23 (d, 1H), 7.70 (t, 1H), 8.00 (d, 1H), 8.92 (s, 1H). Mass Spectrum MH+ 462

6-methyl-2-methylaminopyridine was prepared using the following procedure:

A mixture of 2-chloro-6-methylpyridine (2.56 g, 20 mmol) in 50 ml of a 6N solution of methylamine in methanol was heated in a pressure vessel at 140° C. for 48 hours. The resulting mixture was concentrated and the residue was taken in dichloromethane and treated with ammonium hydroxide. The organic layer was washed with brine, dried and evaporated to give the desired compound. NMR Spectrum (500 MHz, CDCl3) 2.37 (s, 3H), 2.88 (s, 3H), 4.58 (bs, 1H), 6.19 (d, 1H), 6.45 (d, 1H), 7.35 (t, 1H).

### EXAMPLE 12

### N-[3,5-di(morpholin-4-yl)phenyl]-N′-methyl-N′-(5-methylpyridin-2-yl)pyrimidine-2,4-diamine

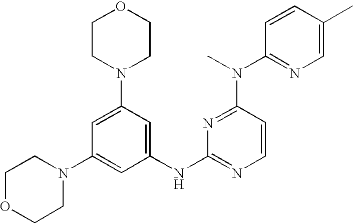

Prepared following the same procedure as above using 5-methyl-2-methylaminopyridine. NMR Spectrum (500 MHz, DMSO-d6) 2.30 (s, 3H), 3.01 (m, 8H), 3.49 (s, 3H), 3.69 (m, 8H), 6.10 (s, 1H), 6.22 (d, 1H), 6.92 (d, 2H), 7.35 (d, 1H), 7.64 (dd, 1H), 7.99 (d, 1H), 8.30 (d, 1H), 8.91 (s, 1H). Mass Spectrum MH+ 462

5-methyl-2-methylaminopyridine was prepared as follows:

A mixture of 2-chloro-5-methylpyridine (2.56 g, 20 mmol) in 50 ml of a 6N solution of methylamine in methanol was heated in a pressure vessel at 140° C. for 48 hours. The resulting mixture was concentrated and the residue was taken in dichloromethane and treated with ammonium hydroxide. The organic layer was washed with brine, dried and evaporated. The crude product was purified on silica gel eluting with 2% to 6% 6N NH3/MeOH in dichloromethane to give the desired compound. NMR Spectrum (500 MHz, CDCl3) 2.17 (s, 3H), 2.89 (d, 3H), 4.42 (bs, 1H), 6.33 (d, 1H), 7.26 (dd, 1H), 7.91 (s, 1H).

### EXAMPLE 13

### [5-[[2-[[3,5-di(morpholin-4-yl)phenyl]amino]pyrimidin-4-yl]-methylamino]-6-methylpyridin-3-yl]methanol

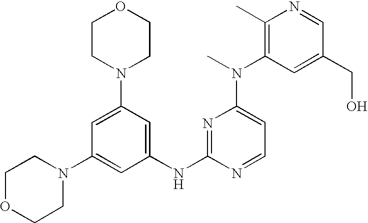

A suspension of (2-chloro-5-((2-(3,5-dimorpholinophenylamino)pyrimidin-4-yl)(methyl)amino)-6-methylpyridin-3-yl)methanol (70 mg, 0.13 mmol), 10% palladium on carbon (10 mg, 0.05 mmol) and potassium carbonate (18.4 mg, 0.13 mmol) in ethanol (9 ml) and water (1 ml), was hydrogenated under 40 psi (2.7 bar) at 20° C. for 4 hours. The resulting suspension was filtered through a pad of celite and the filtrate was concentrated to dryness, diluted with ethyl acetate, washed with a saturated aqueous solution of NaHCO₃, dried over magnesium sulphate and concentrated. The crude product was purified by flash chromatography on silica gel eluting with 0 to 10% methanol in dichloromethane. The solvent was evaporated to dryness, the solid was taken up into diethyl ether and concentrated to afford the title compound (46 mg, 70%) as an off-white solid. NMR Spectrum (500 MHz, DMSO-d6) 2.28 (s, 3H), 3.01-3.09 (m, 8H), 3.36 (s, 3H), 3.68-3.76 (m, 8H), 5.54 (d, 2H), 5.21 (t, 1H), 5.41 (bs, 1H), 6.09 (s, 1H), 6.96 (s, 2H), 7.89 (s, 1H), 7.86 (d, 1H), 8.43 (s, 1H), 8.67 (bs, 1H). Mass Spectrum MH+ 492

The starting material (2-chloro-5-((2-(3,5-dimorpholinophenylamino)pyrimidin-4-yl)(methyl)amino)-6-methylpyridin-3-yl)methanol was prepared as follows:

A solution of lithium aluminium hydride 1M in THF (4.8 ml, 4.8 mmol) was added dropwise to a stirred solution of 5-amino-2-chloro-6-methylnicotinic acid (Journal of Organic Chemistry, 1961, vol. 26, p. 3420; 300 mg, 1.61 mmol) dissolved in THF (10 ml) at 0° C. under argon. The resulting solution was stirred at reflux for one hour. The reaction mixture was cooled in an ice bath and water (0.6 ml) then 2N aqueous sodium hydroxide (0.6 ml) and water (1.2 ml) was added carefully in sequence in order to precipitate the aluminium salts. Ethyl acetate was added and the insoluble material was removed by filtration and washed with ethyl acetate. The organic phase was washed with brine, dried and concentrated to afford the crude product. Purification by flash chromatography on silica gel eluting with 0 to 5% methanol in dichloromethane afforded (5-amino-2-chloro-6-methylpyridin-3-yl)methanol (185 mg, 66%) as a white solid.

A solution of the above (5-amino-2-chloro-6-methylpyridin-3-yl)methanol (180 mg, 1.04 mmol), 4-chloro-N-(3,5-dimorpholin-4-ylphenyl)-N-[(4-methoxyphenyl)methyl]pyrimidin-2-amine (described in Example 3, 517 mg, 1.04 mmol) and hydrochloric acid 4M in dioxane (0.013 mL, 0.05 mmol) in iPrOH (4 mL) was stirred at reflux for 6 hours. The reaction mixture was concentrated to dryness and taken up into a 5:95 mixture of methanolic ammonia 7N and dichloromethane (20 mL). The precipitate was removed by filtration and the filtrate was concentrated. The crude product was purified by flash chromatography on silica gel eluting with 0 to 10% methanol in dichloromethane. Evaporation of the solvent afforded (2-chloro-5-(2-((3,5-dimorpholinophenyl)(4-methoxybenzyl)amino)pyrimidin-4-ylamino)-6-methylpyridin-3-yl)methanol (650 mg, 99%) as a white foam.

Dimethyl sulfate (0.079 mL, 0.84 mmol) was added dropwise to a mixture of (2-chloro-5-(2-((3,5-dimorpholinophenyl)(4-methoxybenzyl)amino)pyrimidin-4-ylamino)-6-methylpyridin-3-yl)methanol (480 mg, 0.76 mmol) and cesium carbonate (742 mg, 2.28 mmol) in DMF (2 mL) at room temperature under argon. The resulting suspension was stirred at 90° C. for 3 hours. The insoluble was removed by filtration and the filtrate was concentrated. The crude product was purified by flash chromatography on silica gel eluting with 0 to 10% methanol in dichloromethane/EtOAc (1/1). The solvent was evaporated to dryness to afford (2-chloro-5-((2-((3,5-dimorpholinophenyl)(4-methoxybenzyl)amino)pyrimidin-4-y)(methyl)amino)-6-methylpyridin-3-yl)methanol (230 mg, 46.9%) as a white foam. This compound (200 mg, 0.31 mmol) and anisole (0.101 mL, 0.93 mmol) were dissolved in TFA (1.5 mL) and sealed into a microwave tube. This mixture was heated to 140° C. over a period of 45 minutes in a microwave reactor. The reaction mixture was allowed to cool to room temperature, basified with a saturated aqueous solution of sodium hydrogencarbonate and extracted with ethyl acetate. The combined organic phases were washed with brine, dried and concentrated under vacuum. The residue was purified by flash chromatography on silica gel eluting with 0 to 10% methanol in dichloromethane/ethyl acetate (1/1). Evaporation of the solvent afforded (2-chloro-5-((2-(3,5-dimorpholinophenylamino)pyrimidin-4-yl)(methyl)amino)-6-methylpyridin-3-yl)methanol (73.0 mg, 44.8%).

NMR Spectrum (500 MHz, DMSO-d6) 2.26 (s, 3H), 3.04 (bs, 8H), 3.36 (s partially hidden by H2O, 3H), 3.66-3.76 (m, 8H), 4.53 (d, 2H), 5.36 (bs, 1H), 5.59 (t, 1H), 6.10 (s, 1H), 6.97 (bs, 2H), 7.78 (s, 1H), 7.87 (bs, 1H), 8.92 (bs, 1H). Mass Spectrum MH+ 526

1. A compound of formula I 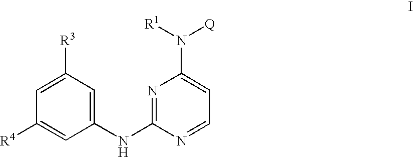
wherein:
R¹ is a (1-4C)alkyl group which is optionally substituted by one or more substituent groups selected from —OR⁵ (wherein R⁵ is selected from hydrogen or (1-2C)alkyl), cyano, halo, or —NR⁶R⁷ (where R⁶ and R⁷ are independently selected from hydrogen, (1-2C)alkyl or (1-2C)alkanoyl);
Q is selected from a group of formula: 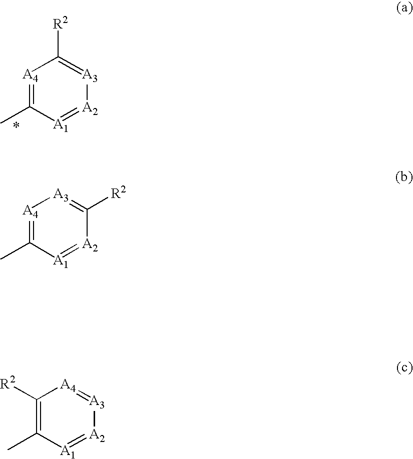
wherein * is the point of attachment to the compound of formula I above;
one of A₁, A₂, A₃, and A₄ is N and the others are —CR^{2a}—;
R² is independently selected from (1-2C)alkyl, (1-2C)alkoxy, fluoro, chloro, cyano, hydroxy(1-2C)alkyl, or a group of sub-formula:
—X¹—R^{y}
where X¹ is selected from —CO—, —NR^{a}—, —NR^{a}—CO—, —NR^{a}—COO—, NR^{a}CONR^{b}, —CONR^{a}—, —S(O)_{z}— (where z is 0, 1 or 2); —SO₂NR^{a}—, and —NR^{a}SO₂—, R^{a} and R^{b} are each independently selected from hydrogen or methyl, and R^{y} is hydrogen or (1-2C)alkyl;
each R^{2a} group present is independently selected from hydrogen, (1-2C)alkyl, (1-2C)alkoxy, fluoro, chloro, cyano, hydroxy(1-2C)alkyl, or a group of sub-formula:
—X²—R^{z}
where X² is selected from —CO—, —NR^{c}—, —NR^{c}—CO—, —NR^{c}—COO—, NR^{c}CONR^{d}, —CONR^{c}—, —S(O)_{z}— (where z is 0, 1 or 2); —SO₂NR^{c}—, and —NR^{c}SO₂—, R^{c} and R^{d} are each independently selected from hydrogen or methyl, and R^{z} is hydrogen or (1-2C)alkyl;
R³ is selected from:
(i) hydrogen, halo, nitro, cyano, or hydroxy;
(ii) an optionally substituted (1-6C)alkyl, (2-6C)alkenyl, or (2-6C)alkynyl group wherein the optional substituents are selected from cyano, halo, or a group of sub-formula:
—W—R⁹
wherein W is selected from —O—, —S(O)ₚ— (where p is 0, 1 or 2), —CO—, —NR^{f}CO—, —CONR^{f}—, —NR^{f}CONR^{f}—, —SO₂NR^{e}, —NR^{e}SO₂—, or —NR^{e}COO—;
R^{e} is selected from hydrogen or (1-2C)alkyl;
and R⁹ is selected from hydrogen or (1-4C)alkyl;
or —NR¹⁰R¹¹, where R¹⁰ and R¹¹ are independently selected from hydrogen, (1-2C)alkanoyl or (1-2C)alkyl, or R¹⁰ and R¹¹ are linked to form a 4, 5, 6 or 7 membered heterocyclic ring which optionally comprises, in addition to the nitrogen atom to which R¹⁰ and R¹¹ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
(iii) a group —NR¹²R¹³, wherein R¹² and R¹³ are each independently selected from hydrogen or (1-6C)alkyl, or R¹² and R¹³ are linked to form a 4, 5, 6 or 7-membered heterocyclic ring which optionally comprises, in addition to the nitrogen atom to which R¹² and R¹³ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl; or
(iv) a group of formula (II):
—X³—R¹⁴
wherein X³ is selected from —O—, —S(O)ₚ— (where p is 0, 1 or 2), —CO—, —NR^{g}CO—, —CONR^{g}—, —NR^{g}COO—, and —NR^{g}SO₂—,
where R^{g} is selected hydrogen or (1-2C)alkyl;
R¹⁴ is a (1-4C)alkyl group which is optionally substituted by halo, hydroxy, cyano, (1-4C)alkoxy, or R¹⁴ is
—NR¹⁵R¹⁶
where R¹⁵ and R¹⁶ are independently selected from hydrogen, (1-2C)alkanoyl or (1-2C)alkyl, or R¹⁵ and R¹⁶ are linked to form a 4, 5, 6 or 7-membered heterocyclic ring which optionally comprises, in addition to the nitrogen atom to which R¹⁵ and R¹⁶ are attached, one or two further heteroatoms selected from O, N or S, and wherein any S atoms that are present may be optionally oxidised to form an SO and SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom is optionally substituted by (1-4C)alkyl, hydroxy(1-4C)alkyl, (1-2C)alkoxy-(1-4C)alkyl, or (1-4C)alkanoyl;
R⁴ is a group —NR¹⁷R¹⁸, wherein R¹⁷ and R¹⁸ are linked to form a 4, 5, 6 or 7 membered heterocyclic ring which optionally comprises, in addition to the nitrogen atom to which R¹⁷ and R¹⁸ are attached, one or two further heteroatoms selected from O, N or S, and wherein any S atoms that are present may be optionally oxidised to form an SO or SO₂ group, and wherein any carbon atom present in the ring is optionally substituted by oxo, halo, hydroxy, cyano, (1-4C)alkyl, hydroxy(1-4C)alkyl, (1-4C)alkoxy, (1-2C)alkoxy-(1-4C)alkyl, (1-4C)alkanoyl, (1-4C)alkanesulfonyl, (1-4C)alkoxycarbonyl, (1-6C)alkylaminocarbonyl or di-(1-6C)alkylaminocarbonyl and any available nitrogen atom present in the ring is optionally substituted by (1-4C)alkyl, hydroxy(1-4C)alkyl, (1-2C)alkoxy-(1-4C)alkyl, or (1-4C)alkanoyl;
or a pharmaceutically acceptable salt thereof.

2. A compound according to claim 1 wherein Q is selected from (a) or (b) as defined in claim 1.

3. A compound according to claim 2 wherein R² is selected from methyl, fluoro, chloro, hydroxymethyl, methoxy, acetamido, or methylthio

4. A compound according to claim 1 wherein each group R^{2a} present is independently selected from hydrogen, methyl, fluoro, chloro, hydroxymethyl, methoxy, acetamido, or methylthio.

5. A compound according to claim 4 wherein one R^{2a} group is other than hydrogen, and the remainder are hydrogen, or all R^{2a} groups are hydrogen.

6. A compound according to claim 1 wherein R¹ is methyl.

7. A compound according to claim 1 wherein R⁴ is a group of formula: 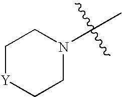
wherein Y is selected from O, S, NR²⁰, or CR²¹, where R²⁰ is selected from hydrogen, (1-2C)alkyl, hydroxy(1-2C)alkyl, (1-2C)alkoxy(1-2C)alkyl, or (1-2C)alkanoyl, and R²¹ is selected from hydrogen, hydroxy, (1-2C)alkyl, hydroxy(1-2C)alkyl, (1-2C)alkoxy(1-2C)alkyl, or (1-2C)alkanoyl,

8. A compound according to claim 1 wherein R³ is a group NR¹²R¹³, wherein R¹² and R¹³ are each independently selected from hydrogen or (1-6C)alkyl, or R¹² and R¹³ are linked to form a 5, 6 or 7-membered heterocyclic ring, and wherein, in addition to the nitrogen atom to which R¹² and R¹³ are attached, the ring optionally comprises one or two further heteroatoms selected from O, N or S, and wherein the ring is optionally substituted on any available carbon atom by one or two substituent groups selected from oxo, halo, hydroxy, cyano, (1-4C)alkyl, or (1-4C)alkanesulfonyl, and any available nitrogen atom present in the ring is optionally substituted by (1-4C)alkyl or (1-4C)alkanoyl

9. A compound according to claim 1 which is selected from:
N-(3,5-dimorpholin-4-ylphenyl)-N′-(4-methoxypyridin-2-yl)-N′-methyl-pyrimidine-2,4-diamine;
N′-(4-chloropyridin-2-yl)-N-(3,5-dimorpholin-4-ylphenyl)-N′-methyl-pyrimidine-2,4-diamine;
N′-(2-chloropyridin-4-yl)-N-(3,5-dimorpholin-4-ylphenyl)-N′-methyl-pyrimidine-2,4-diamine;
N′-(5-chloropyridin-3-yl)-N-(3,5-dimorpholin-4-ylphenyl)-N′-methyl-pyrimidine-2,4-diamine;
N′-(6-chloropyridin-2-yl)-N-(3,5-dimorpholin-4-ylphenyl)-N′-methyl-pyrimidine-2,4-diamine;
N-(3,5-dimorpholin-4-ylphenyl)-N′-(6-methoxypyridin-2-yl)-N′-methyl-pyrimidine-2,4-diamine;
N4-(6-chloropyridin-3-yl)-N2-(3,5-dimorpholinophenyl)-N4-methylpyrimidine-2,4-diamine;
N-(3,5-dimorpholin-4-ylphenyl)-N′-methyl-N′-(6-methylpyridin-3-yl)pyrimidine-2,4-diamine;
N-(3,5-dimorpholin-4-ylphenyl)-N′-(5-methoxypyridin-3-yl)-N′-methyl-pyrimidine-2,4-diamine;
N′-(2,5-dimethylpyridin-3-yl)-N-(3,5-dimorpholin-4-ylphenyl)-N′-methyl-pyrimidine-2,4-diamine;
N-(3,5-dimorpholin-4-ylphenyl)-N′-(5-methoxy-2-methyl-pyridin-3-yl)-N′-methyl-pyrimidine-2,4-diamine;
N′-(6-chloro-5-methoxy-pyridin-3-yl)-N-(3,5-dimorpholin-4-ylphenyl)-N′-methyl-pyrimidine-2,4-diamine;
N′-(6-chloro-5-methyl-pyridin-3-yl)-N-(3,5-dimorpholin-4-ylphenyl)-N′-methyl-pyrimidine-2,4-diamine;
N′-(2-chloro-5-methoxy-pyridin-3-yl)-N-(3,5-dimorpholin-4-ylphenyl)-N′-methyl-pyrimidine-2,4-diamine;
(6-((2-(3,5-dimorpholinophenylamino)pyrimidin-4-yl)(methyl)amino)-5-methylpyridin-2-yl)methanol;
N-[3,5-di(morpholin-4-yl)phenyl]-N′-methyl-N′-(6-methylpyridin-2-yl)pyrimidine-2,4-diamine;
N-[3,5-di(morpholin-4-yl)phenyl]-N′-methyl-N′-(5-methylpyridin-2-yl)pyrimidine-2,4-diamine; or
[5-[[2-[[3,5-di(morpholin-4-yl)phenyl]amino]pyrimidin-4-yl]-methylamino]-6-methylpyridin-3-yl]methanol;
or a pharmaceutically acceptable salt thereof.

10. A pharmaceutical composition comprising a compound according to claim 1 in combination with a pharmaceutically acceptable carrier or diluent.

11. A process for preparing a compound of formula (I) which comprises either
(A) reacting a compound of formula (II): 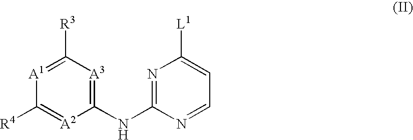
where A¹, A², A³, R³ and R⁴ is as defined in relation to formula I with the proviso that any functional groups are optionally protected, and L¹ is a leaving group, with a compound of formula (III) 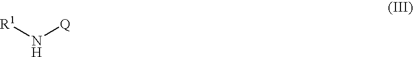
where Q and R¹ are as defined in claim 1 provided that any functional groups are optionally protected; or
(B) by reacting a compound of formula (VII) 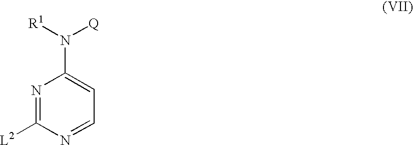
where Q, and R¹ are as defined in claim 1 provided that any functional groups can be optionally protected, and L² is a leaving group, with a compound of formula (VI) 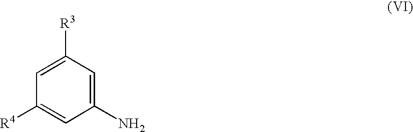
where R³ and R⁴ are as defined in claim 1; or
(C) reacting a compound of formula (XI) 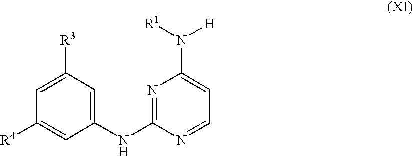
wherein R¹, R³ and R⁴ are as defined above in claim 1;
with a compound of formula (XII)
L⁶-Q (III)
wherein Q is as defined above in claim 1 and L⁶ is halogen, where any functional groups are protected as necessary; or
(D) reacting a compound formula (X) 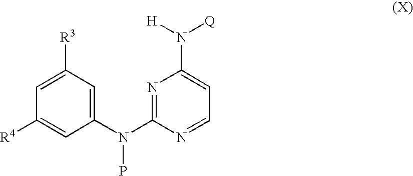
wherein Q, R³ and R⁴ are as defined in claim 1 and P is a suitable protecting group for this reaction, for example a 4-methoxybenzyl group;
with a compound
R¹-L⁷
where L⁷ is a suitable leaving group such as halogen and R¹ is as defined above in claim 1,
thereafter if desired or necessary carrying out one or more of the following steps:
(i) removing any protecting groups, or
(ii) converting a compound of formula (I) obtained into a different compound of formula (I);
(iii) forming a salt.

12. A compound according to for use in the inhibition of an EphB4.

13. A compound according to claim 12 for use in the treatment of cancer.

14. A method of inhibiting EphB4 in a human or animal in need thereof, which method comprises administration of an effective amount of a compound according to claim 1.

15. A method of treating cancer in a human or animal in need thereof, which method comprises administration of an effective amount of a compound according to claim 1.
